(19) United States
(12) Patent Application Publication
(10) Pub. No.: US 2018/0228467 A1
(43) Pub. Date: Aug. 16, 2018

(54) Device, System and Method for Hemispheric Array Breast Imaging

(71) Applicant: University of Rochester, Rochester, NY (US)

(72) Inventor: Robert Waag, Rochester, NY (US)

(21) Appl. No.: 15/431,160

(22) Filed: Feb. 13, 2017

(51) Int. Cl. A61B 8/00; A61B 8/08

(52) U.S. Cl. CPC A61B 8/4494; A61B 8/085; A61B 8/5207; A61B 8/0825; A61B 8/406

(57) ABSTRACT

A device, system, and method for volumetric ultrasound imaging is described. The device and system include an array of transducer elements grouped in triangular planar facets and substantially configured in the shape of a hemisphere to form a cup-shaped volumetric imaging region within the cavity of the hemisphere. A plurality of data-acquisition assemblies are connected to the transducers, which are configured to collect ultrasound signals received from the transducers and transmit image data to a network of processors that are configured to construct a volumetric image of an object within the imaging region based on the image data received from the data-acquisition assemblies. A control module includes a firmware module, a low-level operating-system device driver and an application programming interface library for processes ultrasound signals transmitted and received from the array of transducer elements.

### STATEMENT REGARDING FEDERALLY SPONSORED RESEARCH OR DEVELOPMENT

This invention was made with government support under Grant Nos. EB009692 and EB010069 awarded by the National Institutes of Health (NIH). The government has certain rights in the invention.

### BACKGROUND

Breast cancer is a significant health problem worldwide. In the United States alone, more than 230,000 new cases of invasive breast cancer are estimated to be diagnosed each year and about 40,000 women are expected die of the disease this year. Globally, when excluding non-melanoma cancers of the skin, breast cancer is the most common cancer in women.

An important clinical goal is to detect breast masses when they are as small as possible, preferably less than several millimeters in diameter. Reports indicate that women who have invasive breast cancer detected when the size is less than 15 mm have a 15-year survival rate of 89-93% (95% confidence interval). Imaging is the primary way that cancer in the breast can be detected when the cancer is small. In addition, imaging can also be used for staging and monitoring response to the treatment of a patient with breast cancer.

The breast can be imaged using a number of methods, including conventional x-ray mammography, x-ray tomosynthesis, and magnetic resonance imaging (MRI). However, current implementations of these methods often suffer from low resolution, poor contrast, or other issues that reduce the effectiveness of these techniques in detecting or identifying breast disease.

For example, X-ray mammography is generally considered to be the most cost-effective tool for the early detection of breast cancer. However, the specificity and positive predictive value of mammography is limited, due to the potential overlap in the appearances of benign and malignant lesions, and to poor contrast in patients with dense breast tissue.

Ultrasound is not typically used for the diagnosis of breast disease because the process of obtaining the images is highly operator dependent. Further, ultrasound resolution is generally not adequate, particularly in the direction orthogonal to the imaging plane, i.e., the slice thickness dimension, and speckle can make images hard to interpret or can obscure calcifications. Current ultrasound techniques also often poorly describe lesion margins that are known to be an important feature for the diagnosis of cancer.

Accordingly, there is a continuing need in the art for imaging techniques that can be used to image breasts accurately for the purposes of detecting or identifying breast disease.

### SUMMARY

In one embodiment, a device for volumetric ultrasound imaging is claimed. The device includes an array of ultrasound transducer elements substantially configured in the shape of a hemisphere to form a cup-shaped volumetric imaging region within the cavity of the hemisphere and a control module including a firmware module, a low-level operating-system device driver and an application programming interface library for processes ultrasound signals transmitted and received from the array of transducer elements. In one embodiment, the firmware module is an FPGA firmware module configured to control ultrasound transmissions and receptions, and communicate with a plurality of computing nodes. In one embodiment, the low-level operating-system device driver is configured to run on the computing nodes to enable software interaction with the firmware module. In one embodiment, the application programming interface library abstracts the low-level representation of FPGA hardware by the device driver and provides input validation. In another embodiment, the array of transducers includes 40 triangular planar facets. In another embodiment, 10 of the facets are equilateral triangles and 30 of the facets are isosceles triangles. In another embodiment, each triangular transducers includes 256 piezoelectric elements. In another embodiment, the piezoelectric elements are arranged pseudorandomly on each facet. In another embodiment, at least one of the transducers further includes a diverging lens. In another embodiment, at least one of the transducers further includes two matching layers. In another embodiment, the hemisphere array of transducers is positioned within the surface of a patient table, such that the opening of the cup-shaped volumetric imaging region is substantially flush with the patient table surface. In another embodiment, the device further includes a cup-shaped container sized to fit substantially within the imaging region cavity of the hemisphere. In another embodiment, the cup-shaped container is disposable.

In another embodiment, a system for volumetric ultrasound imaging is claimed. The system includes an array of planar faceted ultrasound transducers substantially configured in the shape of a hemisphere to form a cup-shaped volumetric imaging region within the cavity of the hemisphere, a plurality of data-acquisition assemblies connected to the transducers, a network of processors connected to the data-acquisition assemblies, and a control module including a firmware module, a low-level operating-system device driver and an application programming interface library for processes ultrasound signals transmitted and received from the array of transducer elements. In one embodiment, the firmware module is an FPGA firmware module configured to control ultrasound transmissions and receptions, and communicate with a plurality of computing nodes. In one embodiment, the low-level operating-system device driver is configured to run on the computing nodes to enable software interaction with the firmware module. In one embodiment, the application programming interface library abstracts the low-level representation of FPGA hardware by the device driver and provides input validation. The ultrasound transducers are configured to generate and receive ultrasound signals within the imaging region, the data-acquisition assemblies are configured to collect ultrasound signals received from the transducers and transmit measured data to the network of processors, and the network of processors is configured to construct a volumetric image of an object within the imaging region based on the image data received from the data-acquisition assemblies. In another embodiment, the number of data-acquisition assemblies is equal to the number of transducer elements, and that each data-acquisition assembly is dedicated to an individual transducer. In another embodiment, the array of transducers comprises 40 triangular planar faceted transducer subarrays. In another embodiment, 10 of the facets are equilateral triangles and 30 of the facets are isosceles triangles. In another embodiment, each transducer comprises 256 piezoelectric elements. In another embodiment, each data-acquisition assembly comprises at least 256 send/receive channels. In another embodiment, the network of processors comprises at least 20 nodes. In another embodiment, each node comprises at least one graphical processing unit (GPU). In another embodiment, each node is configured to process data received from at least two data-acquisition assemblies in parallel.

### BRIEF DESCRIPTION OF THE DRAWINGS

The following detailed description of embodiments will be better understood when read in conjunction with the appended drawings. It should be understood, however, that the embodiments are not limited to the precise arrangements and instrumentalities shown in the drawings.

FIG. 1 is a schematic of an exemplary embodiment of a hemispheric breast imaging system (HABIS) integrated with a patient examination table.

FIG. 2 is a schematic of another exemplary embodiment of a HABIS integrated with a patient examination table, wherein the examination table surface and a portion of the sides of the table have been removed.

FIG. 3 is a schematic of the front-end electronics of an exemplary embodiment of a HABIS, showing a hemispheric cup and ultrasound transducer assemblies.

FIG. 4, comprising FIGS. 4A through 4C, is a set of images of an exemplary embodiment of a transducer element subassembly, which can form a part of a hemispheric ultrasound transducer array. FIG. 4A is a photo image of the inner surface of the transducer element subassembly, FIG. 4B is a cross-sectional X-ray image of a portion of the subassembly, and FIG. 4C is a photo image of the outer surface of the subassembly.

FIG. 5 is a schematic of the front-end electronics of an exemplary embodiment of a HABIS, showing a portion of the data-acquisition subassemblies.

FIG. 6 is a schematic of the front-end electronics of an exemplary embodiment of a HABIS, showing a plurality of data-acquisition subassemblies connected to the ultrasound transducer assemblies.

FIG. 7 is a schematic of the front-end electronics of an exemplary embodiment of a HABIS, showing how an exemplary data-acquisition subassembly is connected to an ultrasound transducer assembly.

FIG. 8 is a schematic of the AC wiring and power supply distribution of an exemplary embodiment of a HABIS.

FIG. 9 is a schematic of the HABIS patient examination table of FIG. 1 positioned in an exemplary examination room.

FIG. 10 is a schematic of a hemispheric volume (FIG. 10A) and the tetrahedral region that is associated with a subtriangle of the hemispheric array (FIG. 10B). The portion of the hemispheric volume imaged by the data from the triangular block of receive elements in the triangle is the tetrahedron formed by the vertices of the solid-line triangle and the center of the hemisphere.

FIG. 11A is a schematic of an exemplary embodiment of a high-performance computer network communicatively connected to the hemispheric transducer array of a HABIS.

FIG. 11B is an enlarged view of a portion of the high-performance computer network of FIG. 11A.

FIG. 12 is a diagram of a data processing flow through an exemplary HABIS and associated high-performance computer network.

FIG. 13 is a diagram of a tetrahedral region associated with a subtriangle of the hemispheric array. Image values are computed at vertices of a set of cubic subvolumes with centers that intersects the tetrahedral subvolume.

FIG. 14 is a block diagram of firmware functionality according to one embodiment.

FIG. 15 is a block diagram of a specific implementation of FPGA firmware functionality according to one embodiment.

FIG. 16 is a schematic of a library and driver software stack according to one embodiment.

FIG. 17 is a schematic of a specific implementation of a HABIS API library and driver software stack according to one embodiment.

FIG. 18 is a schematic of an internal structure of the HABIS API library and software stack according to one embodiment.

FIG. 19 is a specific implementation of an internal structure of the HABIS API library and software stack according to one embodiment.

FIGS. 20A and 20B are diagrams of the reconstruction of a 10-point resolution target (14A) and a corresponding b-scan of the target (14B). The surfaces shown are 50% of the maximum amplitude. The two upper-most points are 200 μm apart, the two left-most points are 300 μm apart, and the two right-most points are 400 μm apart. In each panel, an edge of the cubic volume has a length of 2.9 mm and the tic increment on the axes is 0.5 mm.

FIG. 21 is a set of images showing sections of a representative subvolume in a model of the breast. Columns left to right: Sections in the original subvolume, corresponding sections in the inverse-scattering reconstruction of the subvolume, and corresponding sections in the aberration-corrected b-scan of the subvolume. Rows top to bottom: Orthogonal relation between the subvolume sections, x-y plane at z=0, x-z plane at y=0, and y-z plane at x=0. Scattering throughout the hemisphere was used to estimate scattering obtained from a virtual array in the opposite hemisphere and then both sets of data were used in the inverse scattering reconstruction. The b-scan used reception from a 64-element subtriangle and used transmission from that 64-element subtriangle and three surrounding 64-element subtriangles to allow quick formation of a volumetric image. In each panel, an edge of the section has a length of 6.4 mm, and the outer region of the section is weighted by a window used to blend adjacent reconstructed subvolumes. The x, y, and z axes are denoted accordingly. The z axis is the polar axis of the hemisphere, the approximate direction of the b-scan lines which mainly highlight discontinuities perpendicular to them. The original subvolume and the reconstructed subvolume are shown on a linear scale and the b-scan is shown on a log scale.

### DETAILED DESCRIPTION

It is to be understood that the figures and descriptions have been simplified to illustrate elements that are relevant for clear understanding, while eliminating, for the purpose of clarity, many other elements found in the field of ultrasound imaging systems. Those of ordinary skill in the art may recognize that other elements and/or steps are desirable and/or required in implementing the systems and methods described herein. However, because such elements and steps are well known in the art, and because they do not facilitate a better understanding, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art. Any methods and materials similar or equivalent to those described herein can be used in the practice for testing of the systems and methods described herein. In describing and claiming the systems and methods, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “HABIS,” “system,” and the like are used interchangeably herein and refer to a system comprising a hemispheric array of ultrasound transducers and a computer network suitable for high-performance parallel processing of data collected from the hemispheric array. As described herein, such a system may include software and associated algorithms to reconstruct volumetric images of a patient's breast. It is also contemplated herein that such a system can be configured to reconstruct volumetric images of other parts of a patient's anatomy, or any other scattering object.

The terms “patient,” “subject,” “individual,” and the like are used interchangeably herein, and refer to any animal amenable to the systems, devices, and methods described herein. Preferably, the patient, subject or individual is a mammal, and more preferably, a human.

Ranges: throughout this disclosure, various aspects can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

Described herein is a hemispheric breast imaging system (HABIS) that acquires ultrasound scattering measurements using a hemispheric array of transducers, and reconstructs the volume of a subject's breast using a high-performance computer network. As contemplated herein, there are two hardware components of HABIS, i.e., a data-acquisition apparatus and a high-performance computer network, have been integrated to implement efficiently and accurately an inverse scattering algorithm that reconstructs high-resolution images of a subject's breast volume. In one embodiment, the data-acquisition apparatus includes an array of ultrasound transducers arranged in a generally hemispheric pattern and all electronics required for transmitting and receiving ultrasound signals from the array. In one embodiment, the high-performance computer network includes a plurality of interconnected computer nodes configured for fast, parallel processing of the data received from the data-acquisition apparatus.

Described herein is also an inverse scattering algorithm for reconstructing an image of a breast or other target scattering object. The general purpose of the inverse scattering algorithm is to reconstruct an image of the scattering object from measurements of the effects that the object has on incident signals used to probe the object.

However, reconstructions using existing techniques can be challenging for a number of reasons. For example, the relationship between the object and the scattering measurements is nonlinear, thus, high-quality reconstructions generally require iterative procedures. Since only a finite set of scattering measurements can be acquired, the scattering object can never be completely characterized. The scattering information is particularly limited when physical restrictions are present on the range of incident and receive signal directions. Further, fine details of the object correspond to small variations in the scattering measurements, which can be overwhelmed by other scattering responses and can be easily confused with system noise. Lastly, numerical methods that ameliorate these problems can be prohibitively time-consuming, computationally expensive, or both. HABIS resolves the above issues through the implementation of a unique algorithmic and engineering design as described herein.

As described herein, HABIS provides speckle-free, high-resolution, quantitative images of intrinsic tissue characteristics, e.g., sound speed and attenuation slope, for improved detection, diagnosis, and treatment monitoring of breast cancer. In one embodiment, the system can acquire data during an approximately two-second interval by using 10,240 parallel channels for transmission and reception. The system can image an entire breast volume within minutes, with isotropic point resolution as good as the lateral resolution of x-ray mammography, by using an algorithm that independently and simultaneously reconstructs subvolumes spanning the breast. Using ultrasound, the system can be used to examine a breast with non-ionizing radiation for cancer detection. The system overcomes limitations of x-ray mammography such as low resolution of contrast in dense breast tissue, i.e., breast tissue with high x-ray attenuation; distortion and discomfort resulting from compression-induced deformation of the breast, and poor imaging of breasts having implants. Accordingly, the system can significantly improve the detection and diagnosis of breast cancer, and can also improve the monitoring of response to breast cancer treatment, compared to systems currently available.

The reconstruction algorithm described herein can reconstruct subvolumes independently, i.e., in parallel. Graphic processing units (GPUs) allow small high-performance computers (HPCs) to perform massively parallel computations that are ideally suited for implementation of the described parallelized reconstruction algorithm. Accordingly, in one embodiment, the system can include a GPU-based HPC network coupled to a data-acquisition apparatus that enables reconstructions of the breast volume to be obtained in a relatively short amount of time. For example, in one embodiment, the breast volume can be reconstructed in less than 20 minutes, and in other embodiments, in less than 15 minutes, in less than 10 minutes, or in less than 5 minutes. However, it is contemplated herein that a person skilled in the art could readily modify the system to even further decrease the reconstruction time, for example by using faster processing units or by increasing the number of nodes. In contrast to this, on a supercomputer using an existing method for reconstruction of a large single volume, such reconstructions can require time periods of as much as a few days. This improvement in reconstruction time provided by HABIS can have an enormous impact on the clinical utility of imaging systems because data can be acquired, and the reconstructed volumes of the breast viewed, in the course of a single visit. Accordingly, because HABIS is capable of producing images with at least 100 micron resolution in minutes, HABIS provides an improved and efficient way to screen for breast cancer and also to diagnose other breast diseases. In a manner previously unattainable.

### Imaging System

In one embodiment, HABIS comprises an apparatus for acquiring data from an array of ultrasound transducers. In another embodiment, HABIS includes an examination table that houses or otherwise integrates at least part or all of the data acquisition apparatus, which may include both its front-end and back-end electronics. In yet another embodiment, the data acquisition apparatus can be connected to a network of computers that can process or otherwise perform the desired image reconstruction. For example, in one embodiment, the front-end electronics are arranged radially under the patient “head” end of the table around the generally hemispheric transducer array. In one embodiment, under the foot-end of the table are electronic modules that can include power supplies, power-supply filter boards, control boards, isolation transformers, high-voltage regulator boards, circuit breakers, and cable channels, as needed. In one embodiment, the system can also include other components suitable for use with a particular application associated with the image acquisition, such as a fluid-handling cart and an operator console which can be positioned near the examination table.

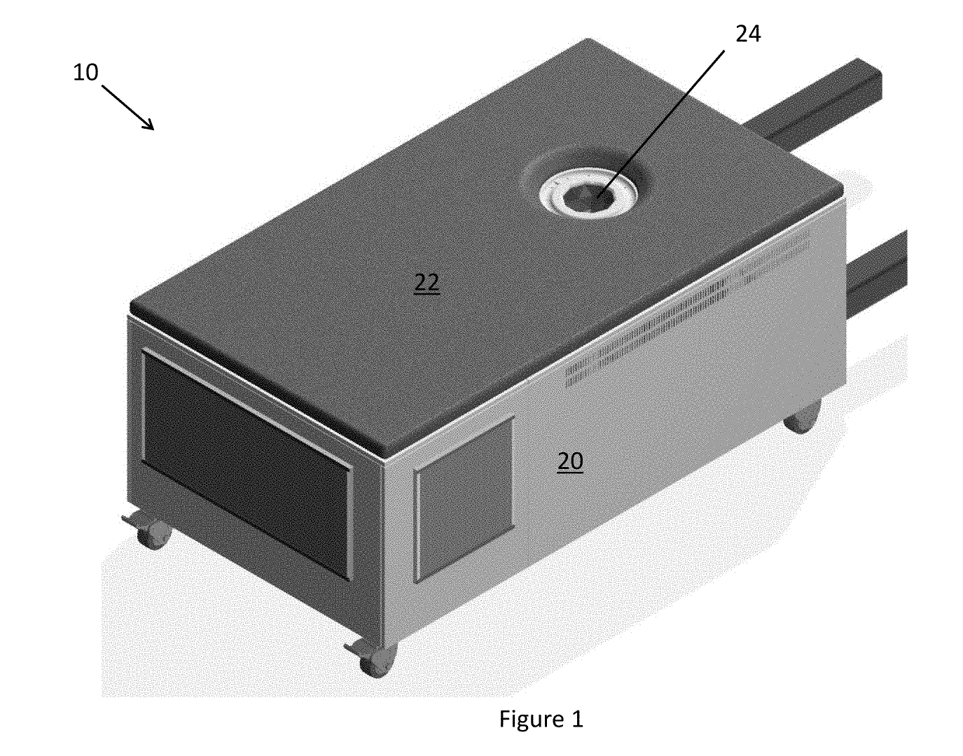

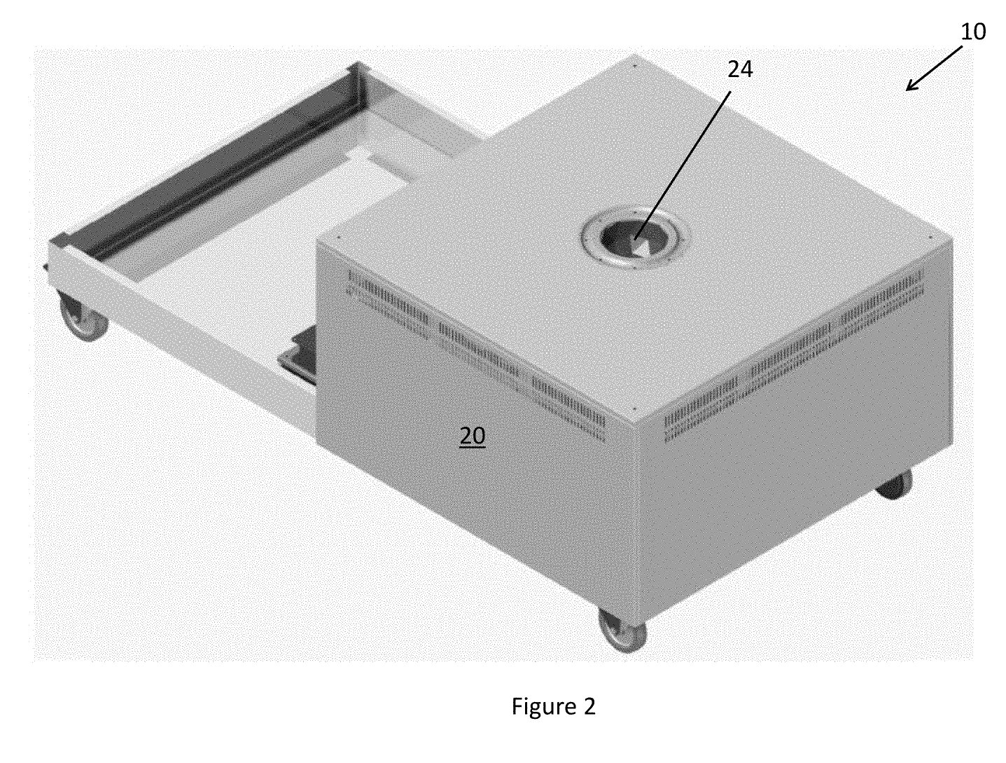

Referring now to FIG. 1, an exemplary embodiment of the data acquisition apparatus 10 of the system is shown, wherein the apparatus is at least partially integrated into the surface of an examination table 20 generally having a surface 22 for a subject to lie on. Surface 22 has an opening or cup 24 into which a scattering object, such as a patient's breast, can be placed. The hemispheric transducer array of the system may form the exposed walls of cup 24, or alternatively, cup 24 may be a permanent or disposable container that fits within the cavity of the transducer array hemisphere to prevent direct contact of the scattering object with the transducers, and/or to hold a suitable amount of coupling fluid, gel or other material used during the imaging procedure. For example, the container may be a detachable and disposable cup-shaped container. Accordingly, a patient can lie prone on table surface 22 with either the right breast or the left breast pendant in cup 24. Further, cup 24 can be filled with a coupling fluid or any other suitable material as desired during an ultrasound imaging procedure. FIG. 2 is a schematic of another embodiment of data acquisition apparatus 10, wherein the examination table surface and a portion of the sides of table 20 have been removed. Again, the head end of table 20 houses the hemispheric array assembly, which is designed to image an object placed in cup 24.

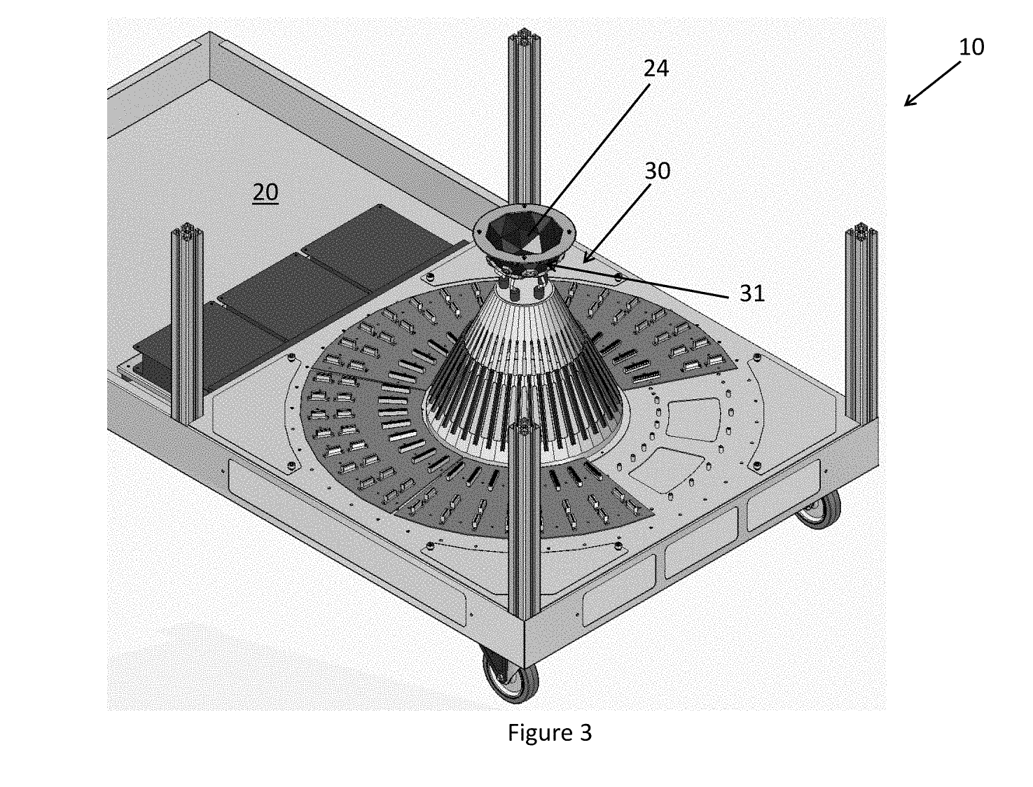

FIG. 3 is another schematic of data acquisition apparatus 10, wherein all sides of table 20 have been removed to show a portion of a hemispherical array assembly 30. Hemispherical array assembly 30 includes a plurality of transducers, or transducer element assemblies 31 having an inner surface for transmitting and receiving ultrasound signals via a plurality of piezoelectric elements, and an outer surface configured with suitable circuitry for transferring electrical current and signal data to/from the piezoelectric elements within the transducer facets. Accordingly, the inner surface of cup 24 forms a cavity for holding a scattering object, for example a patient's breast, that can be imaged using ultrasound. Further, cup 24, or otherwise the cavity formed by the inner surfaces of the transducers, also defines the imaging region within the apparatus or device. As contemplated herein, the cup can comprise any material suitable for ultrasound analysis, as would be understood by a person skilled in the art. In the embodiments described herein, the cup is substantially hemispherical in shape. However, the cup may be a shape other than a hemisphere, provided that the array of ultrasound transducers and other electronic components of the system, and also the image reconstruction algorithm described later herein, are suitably modified to suitably image the resultant object volume within the imaging region.

In one embodiment, data acquisition apparatus 10 of the system can acquire a complete scan of over 100 million receive waveforms in about a two-second time period, with minimal noise contributions to the receive waveforms, and with very precise control over both the timing of the transmissions and the sampling of the received waveforms. In various embodiments, hemispheric array assembly 30 comprises a plurality of data-acquisition subassemblies connected to the transducer elements. In one embodiment, hemispheric array assembly 30 comprises forty (40) data-acquisition subassemblies that are each connected to a single transducer element assembly. However, the number of data-acquisition subassemblies is not limited to forty, and can be more or less than forty, depending on the number of transducer assemblies and/or other factors, such as the desired resolution and/or speed of the reconstructed image.

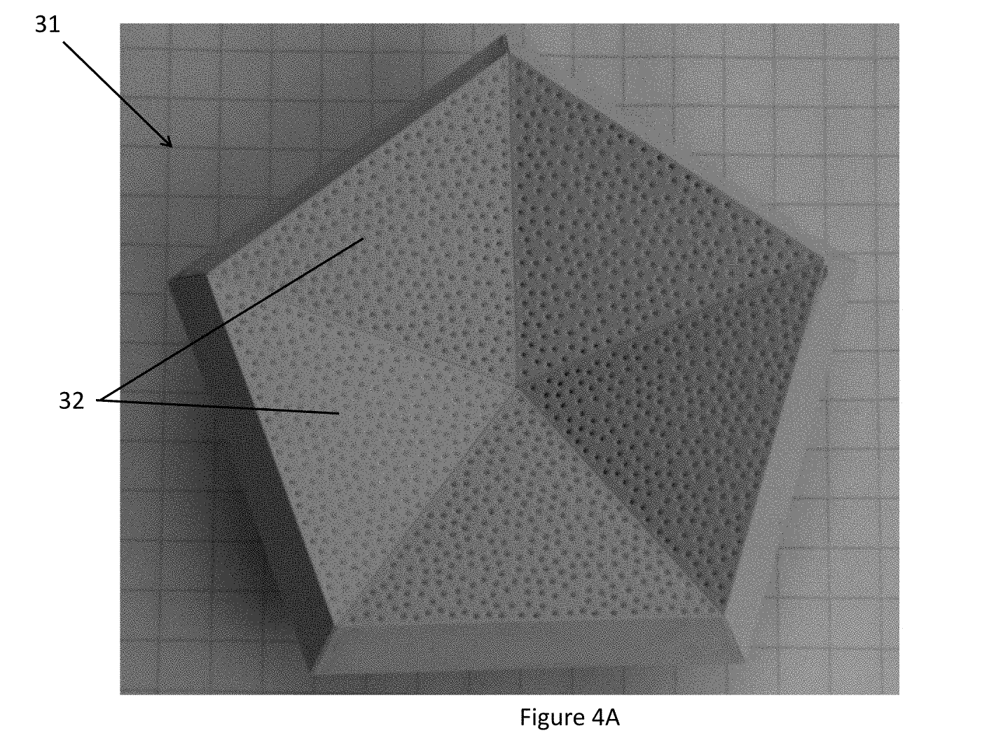

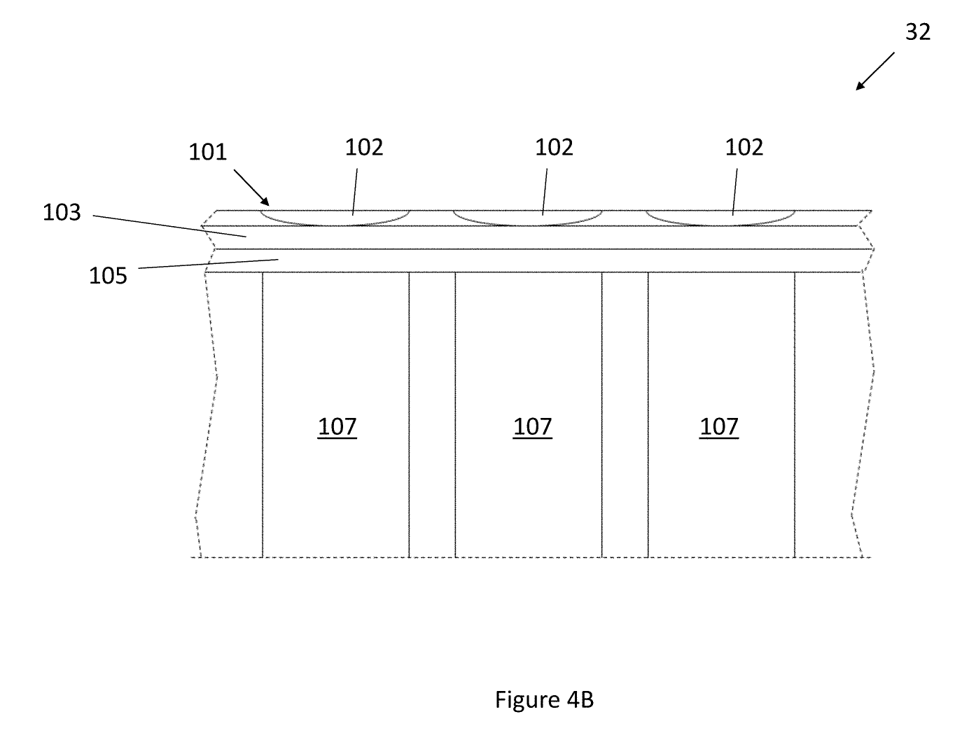

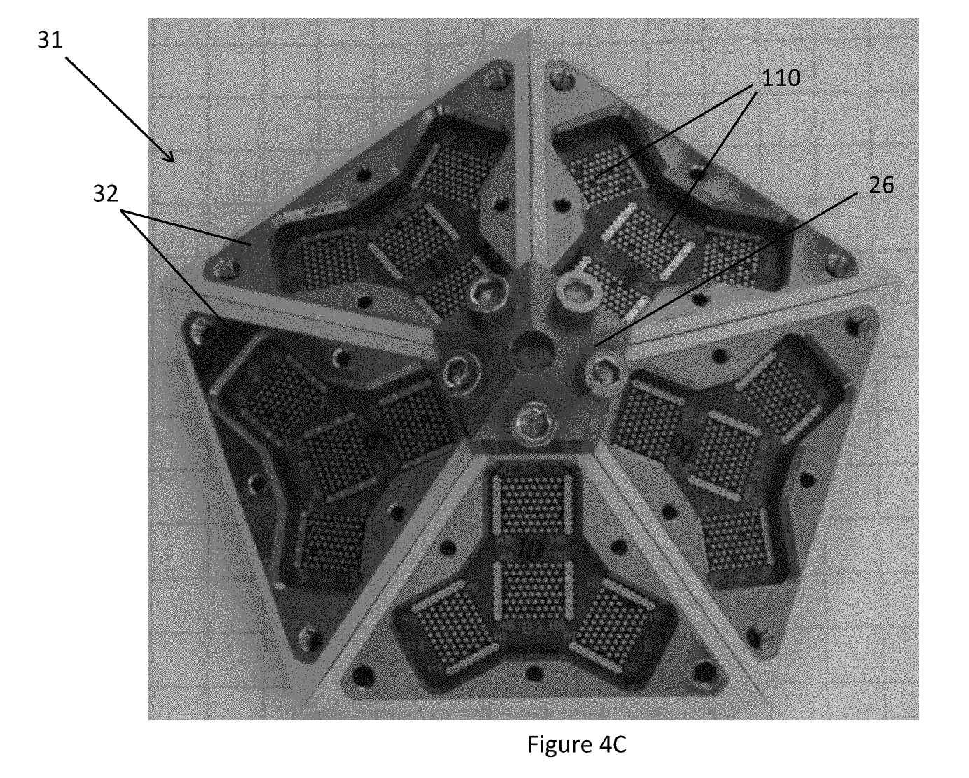

Referring now to FIG. 4, an exemplary embodiment of a transducer element subassembly 31 is shown. FIG. 4A is a view of the inner surface of transducer element subassembly 31 comprising five triangular planar facets 32. Each planar facet 32 comprises a plurality of piezoelectric elements that are located underneath the dimples or depressions on the surface of facets 32. In FIG. 4B, a cross-sectional X-ray of an exemplary planar facet 32 is shown. Facet 32 includes a surface layer 101 that comprises a plurality of depressions or dimples 102. In one embodiment, a diverging lens is located in each dimple 102. In one embodiment, surface layer 101 can be coated with a polymer, for example a Parylene polymer. Beneath surface layer 101 are two matching layers 105. In one embodiment, facet 32 can comprise a number of matching layers other than 2, for example 1 or 3 matching layers. Matching layers 103 and 105 can comprise any material, and can be any thickness, that would be suitable for an ultrasound transducer matching layer, as would be understood by a person skilled in the art. Beneath matching layers 103 and 105 is a plurality of ultrasound transducer elements 107. Each transducer element 107 is substantially aligned with a dimple 102. Transducer elements 107 can comprise any material suitable for an ultrasound transducer known in the art, for example PZT crystal. Further, facet 32 comprises a backing layer (not shown) beneath transducer elements 107. The backing layer can comprise any suitable backing material for an ultrasound transducer. In one embodiment, the backing layer comprises a conductive epoxy. In one embodiment, facet 32 can comprise additional layers, for example a conductive metal coating on either side of the transducer elements, for grounding and excitation. In one embodiment, facet 32 can comprise additional components, for example printed circuit boards. FIG. 4C is a view of the outer surface of transducer subassembly 31. Five triangular planar facets 32 are connected together via a bracket 26. A plurality of wire connectors 110 are located on the outer surface of each facet 32. Wire connectors 110 can be used to communicatively couple transducer elements 107 to the data-acquisition components described later herein, for example via printed circuit boards.

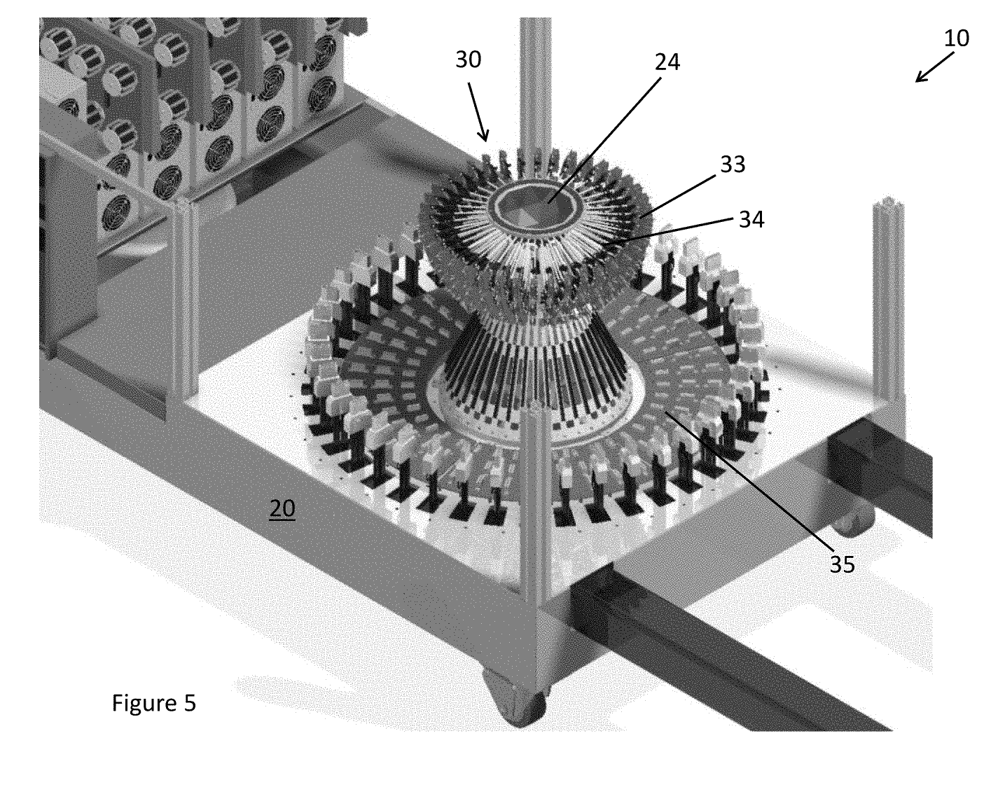

Referring now to FIG. 5, transducer assemblies 31 of hemispheric array assembly 30 are shown coupled to forty front-end boards, i.e., data-acquisition subassemblies 33 (only a portion of these boards are shown in FIG. 5). In this embodiment, data-acquisition subassemblies 33 radiate from the axis of assembly 30. In one embodiment, each data acquisition subassembly 33 comprises 256 channels of electronics, and is connected to the transducer assemblies 31 via cable connections 34, which may be relatively short in length to minimize signal loss. In one embodiment, cable connections 34 are PCIe cables. Hemispheric array assembly 30 also includes bottom-plane boards 35 for distributing power and timing, as would be understood by those skilled in the art. In one embodiment, data collected by at least one of the 256-channel data acquisition subassemblies 33 can be sent to imaging computers through suitable cables, e.g., PCIe cables. In one embodiment, the cables may be about five meters long, but it should be appreciated that these cables can be any length desired, as would be understood by a person skilled in the art. This arrangement allows each ultrasound transducer element assembly 31 to be in close proximity with the front-end circuitry that is used to collect signals from the element. The close proximity between the transducer elements and the data acquisition circuitry lowers signal loss and extends the range and angle over which useful signals can be transmitted and received.

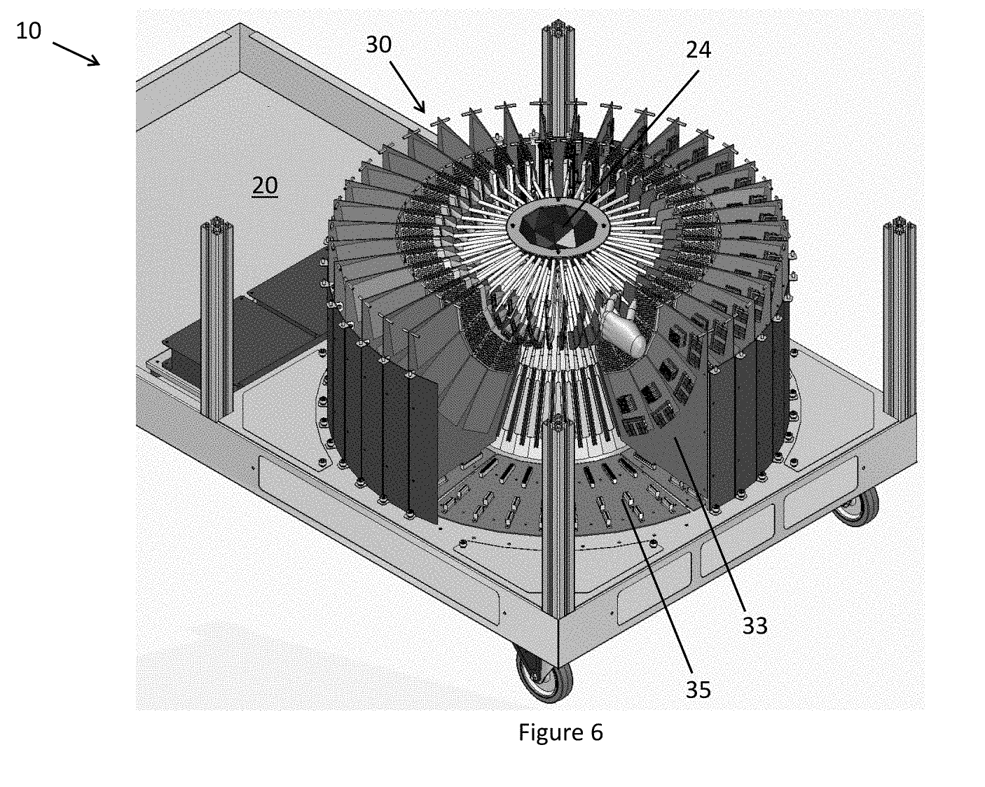

Referring now to FIG. 6, the forty sets of data-acquisition electronics subassemblies 33 are shown mounted in groups of eight on five bottom plane boards 35 that together form an annulus encircling the axis of the hemisphere array in a plane below the hemisphere. Bottom-plane boards 35 are used to distribute timing and control information as well as to provide power to the forty data-acquisition subassemblies 33. This data-acquisition subassembly and bottom-plane arrangement enables high-precision timing signals to be distributed without appreciable degradation. In the embodiment shown, each data-acquisition subassembly 33 includes four blocks of 64-channel electronics positioned in successive angular sections of the board. This arrangement allows transducer assemblies 31, comprising a total of 10,240 transducer elements, to be in proximity to the hemispheric array electronics. In such an embodiment, transducer assemblies 31 are connected to the electronics through 160 short cables.

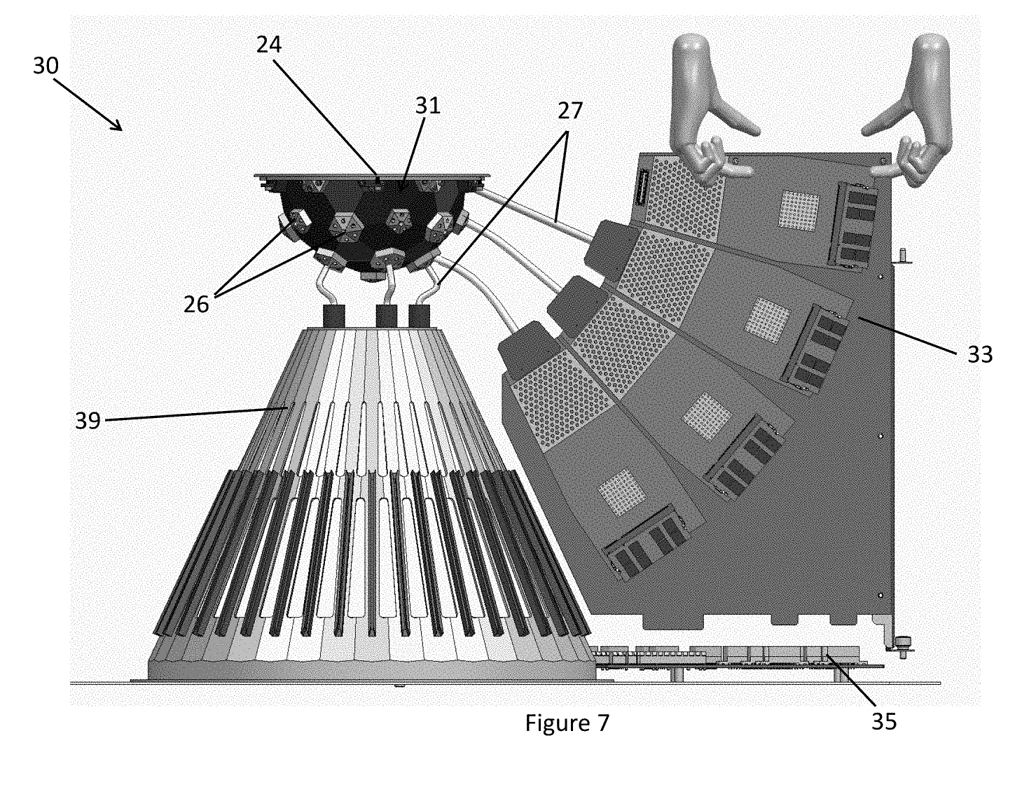

FIG. 7 shows how the 256-channel data-acquisition subassemblies 33 plug into hemispheric array assembly 30 and shows the conical frame 39 that supports the inboard edge of the 256-channel subassemblies 33. Conical frame 39 also forms a plenum to direct air flow, for example air flow from fans that can be placed in another portion of the examination table. In one embodiment, each transducer element subassembly 31 includes a bracket 26. In one embodiment, brackets 26 can be connected to data-acquisition subassemblies 33 via a plurality of flexible supports 27. Further, transducer element assemblies 31 are connected to data-acquisition subassemblies 33 via wiring suitable for sending and receiving signals (not shown).

In one embodiment, during each two-second scan, every data acquisition subassembly 33 receives 10,240 waveforms with 4,096 temporal samples in each waveform from 256 different transducer elements located in transducer assemblies 31. Thus, a total of 32.2 GB is acquired by each subassembly. By comparison, if a typical computational system would be used for reconstruction, then the data from all forty data acquisition subassemblies 33, i.e., a total of 40×32.2 GB, or 1.28 TB, would need to be aggregated and transferred to that system. However, a significant part of the processing can be performed on data from each acquisition subassembly 33 without reference to data from the other acquisition subassemblies. As a result, the amount of data that needs to be transferred to a computer system to complete the reconstruction, after the initial phase of independent parallel processing has been completed, is about 1,000 times smaller than without the use of such parallel processing. Currently available GPU architectures have the processing capability to complete the computations required for HABIS. Accordingly, data acquisition apparatus 10 includes GPU cards that are distributed in the nodes of the data acquisition network. Further, superimposing a web of InfiniBand connections on the network enables rapid inter-node communication necessary in later stages of the computation so that the remainder of the reconstruction computations can be completed with similar speed. As a cost-saving and space-saving measure, in one embodiment, the HABIS data-acquisition subassemblies each incorporate 256 channels of circuitry. Such an arrangement can be used to prevent noise from the digital electronics from interfering with analog reception.

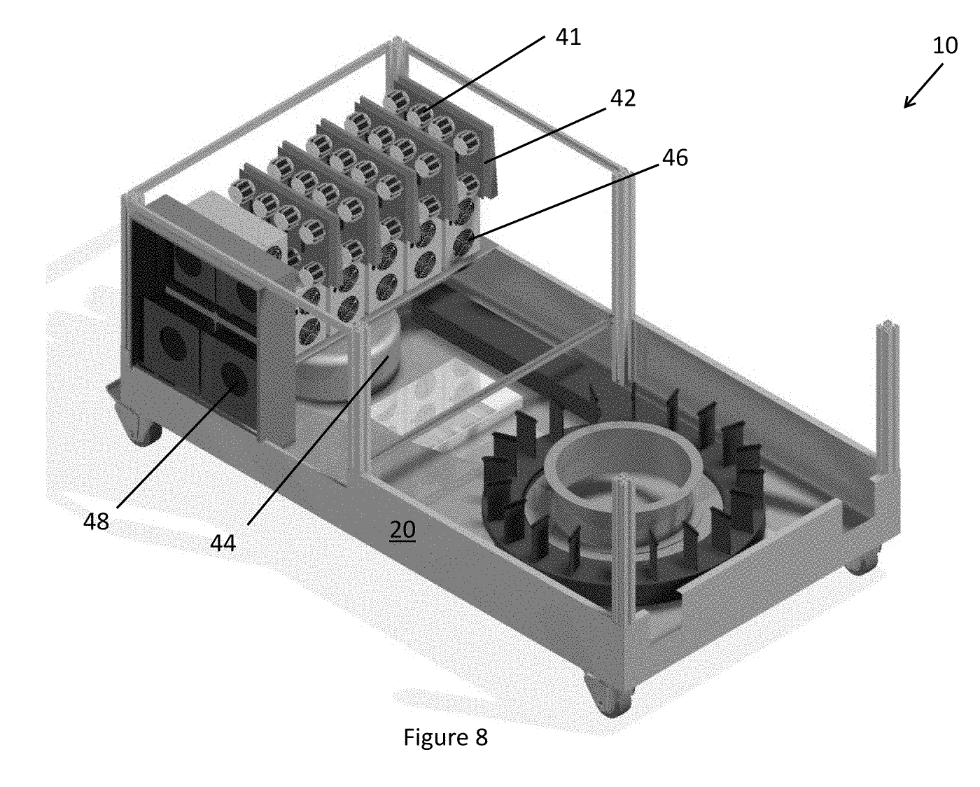

FIG. 8 is a diagram showing the power supply assembly 40 positioned within the interior of table 20. Power supply assembly 40 can include components necessary for the operation of hemispheric array assembly 30, including, but not limited to: a plurality of power supplies 41, power-supply filter boards 42, isolation transformers 44, cooling fans 46, air ducts 48, high-voltage regulator boards, a power-control module, wiring connection blocks, disconnect switches, and circuit breakers within examination table 20. Alternatively, in one embodiment, such components can be placed in a location outside of examination table 20, for example in a secondary cabinet.

FIG. 9 is a diagram showing examination table 20 in an examination room. Examination table 20 can be connected to an operator console 51, which can be used to control any portion of the system, as needed. In addition, a fluids cart 50 can be connected to table 20 to provide a supply and/or recirculation of coupling, i.e., ambient fluid to cup 24. Further, table 20 can be connected to a computer network/scalable server set 70 for image reconstruction, as will be described later herein.

In various embodiments, the components of HABIS can be connected to one or more power supplies suitable to provide power for their operation. For example, in one embodiment, examination table 20 can be connected to a three-phase 208-volt AC supply through a disconnect switch and an isolation transformer. Computer network 70 can be connected to another three-phase 208-volt AC supply through another disconnect switch. In one embodiment, no isolation transformer is included in computer network 70 because the only connection between examination table 20 and computer network 70 is a set of 40 low-voltage PCIe cables. However, the various components of HABIS can be supplied power in any manner as would be understood by a person skilled in the art, and the power supply arrangement is not limited to the specific embodiments described herein.

As previously described, in one embodiment, HABIS is an integrated data-acquisition apparatus and high-performance network that rapidly collects imaging data and efficiently implements fast parallel computation of images via an inverse scattering algorithm. In such an embodiment, the architecture of the system permits GPU-based parallel computations on each node and InfiniBand-based aggregation of results at each node. The design of the system avoids extensive time-consuming data aggregation, permits parallel computation of refined data sets that substantially reduce the amount of data transferred between computer nodes for subsequent parallel reconstruction of subvolumes, and enables fast transfer of intermediate computational results between computing nodes. The parallel computation takes place on two levels: a high level on each computing node and a low level on each GPU. A head node in the network can provide command, control, and monitoring. In one embodiment, an Internet connection can enable command, control, and monitoring to be performed remotely. Data-acquisition apparatus 10, as shown and described herein, provides a large number of independent channels, which can accommodate a large volume of data in a short time period, thereby exceeding the performance of currently available systems. For example and without limitation, the system architecture may include separate connections between pairs of data-acquisition electronics sets and nodes of the high-performance computer network. These connections allow processing to take place prior to aggregation of the receive signals. The system architecture may also include configuration of individual compute nodes with cost-efficient resources that efficiently perform the parallel computations used in HABIS reconstructions. The system architecture may also include InfiniBand connections and switching that efficiently perform in a cost-effective manner the burst transfers of data after each stage of computation. The system architecture may also include a mechanical configuration of the data-acquisition apparatus to minimize the lengths of the data paths between the transducer elements and the transmit and receive electronics so that the range and angle over which useful signals can be transmitted and received is greatly extended. The system architecture may also include circuitry designs to reduce cost by consolidation of data-acquisition and data processing electronics. Further, the system architecture may also include a timing and control system that meets the stringent tolerances imposed by coherent imaging and transmission encoding.

### Transducer Elements and the Hemispheric Array

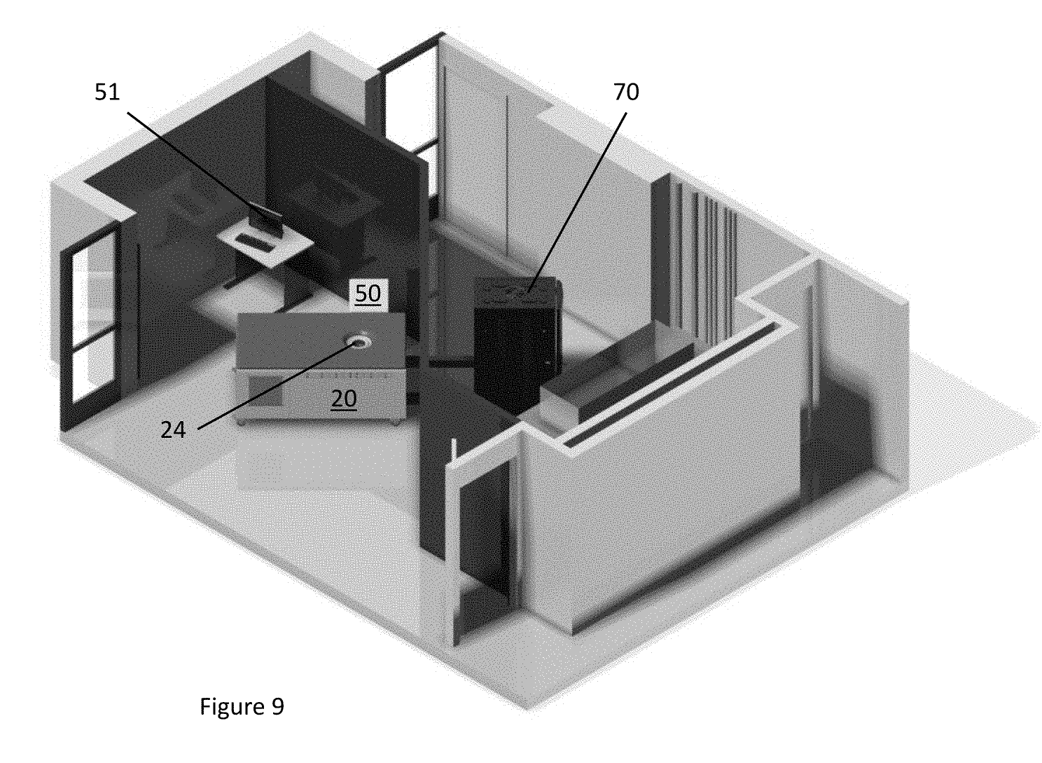

In one embodiment, the array of transducer elements of HABIS is arranged in a faceted construction to provide imaging of a generally hemispheric volume. As shown in FIG. 10, in one embodiment, the hemispheric array in HABIS is approximated using 40 triangular planar facets. In one such embodiment, ten of the facets are equilateral triangles and 30 of the facets are isosceles triangles. The use of a faceted approximation of a hemisphere with two types of triangular planar elements greatly simplifies the construction of the array. Further, in such an embodiment, each facet contains 256 small diameter piezoelectric elements, as indicated by the dots on the facets in FIG. 10A, made from a ceramic material composite that suppresses undesirable lateral modes of vibration.

In one embodiment, the elements on the facets are positioned pseudorandomly. The pseudorandom positions permit the use of many fewer elements (and, thus, fewer independent transmit and receive channels) than would be otherwise required to avoid grating lobes during the formation of transmit and receive beams. In one embodiment, one configuration of pseudorandom positions is used on the equilateral facets and another configuration of positions is used on the isosceles facets. The use of two configuration sets of pseudorandom positions significantly facilitates practical fabrication of the array without degrading the capability of the array to form beams.

Each element may include a diverging lens that broadens the pattern of the transmit beam and the pattern of the receive sensitivity. This broadening extends the volume, i.e., the solid angle, of the scattering object illuminated by transmissions from the elements. The broadening also extends the volume i.e., the solid angle, of the scattering object from which ultrasound signals can be received by the elements. The result of the extended coverage is an appreciably enhanced capability to concentrate focuses formed using the array.

Each element in the array may include two matching layers. The matching layers produce a wider temporal-frequency bandwidth than would be obtained without the layers. The matching layers also increase the transmission energy over the energy that would be transmitted without the layers. Additionally, the layers increase the sensitivity of reception over the reception sensitivity that would be obtained without the layers. Further, the transmit waveform applied to the transducer elements is designed to concentrate the transmitted energy within the temporal-frequency bandwidth of elements.

In addition, the receive circuitry contains a tuning element, i.e., an inductor, that cancels the bandwidth-narrowing effect of the capacitance associated with the transducer elements, the cable between the transducer element and the front-end electronics, the parasitic capacitance of the printed circuit wiring, and the input capacitance of the low-noise amplifier in the receiver chain.

The use of transmissions that are coded without degrading crosstalk and the ability to decode receptions without degrading crosstalk is enabled by the front-end electronics assembly design of HABIS. In the system, the connection between the transducer elements and the cable to the electronics is made through a special signal redistribution arrangement implemented on a printed circuit board with wide trace separation and shielding. Additionally, a special pattern of shielded connections between the cable from the transducer and printed circuit board containing the front-end electronics reduces crosstalk significantly compared to the crosstalk that would otherwise exit. The tuning elements noted above are shielded and widely separated to suppress magnetic coupling between channels.

### High-Performance Computer Network

As previously described, in one embodiment, HABIS includes a high-performance computer network that is connected to the data-acquisition assembly. A specialized architecture consisting of a data-acquisition apparatus and a high-performance computer network is required to collect the ultrasound data in a short time and to reconstruct the breast volume in minutes thereafter. This is because a computational capability must be integrated into HABIS to enable immediate parallel preprocessing of the acquired data. The initial computations performed in the data-acquisition subassemblies yield refined data sets that substantially reduce the amount of data transferred between computer nodes for subsequent parallel reconstruction of subvolumes. As mentioned elsewhere herein, the system architecture may include 40 sets of data-acquisition subassemblies, with each set containing 256 independent transmit and receive channels, and 20 high-performance computing nodes with each node containing four GPUs. In other embodiments, comparable architectures with different numbers of data-acquisition subassemblies and high-performance computing nodes can be used, as the system is not limited to the specific numbers and arrangements of data-acquisition subassemblies and computer nodes described herein.

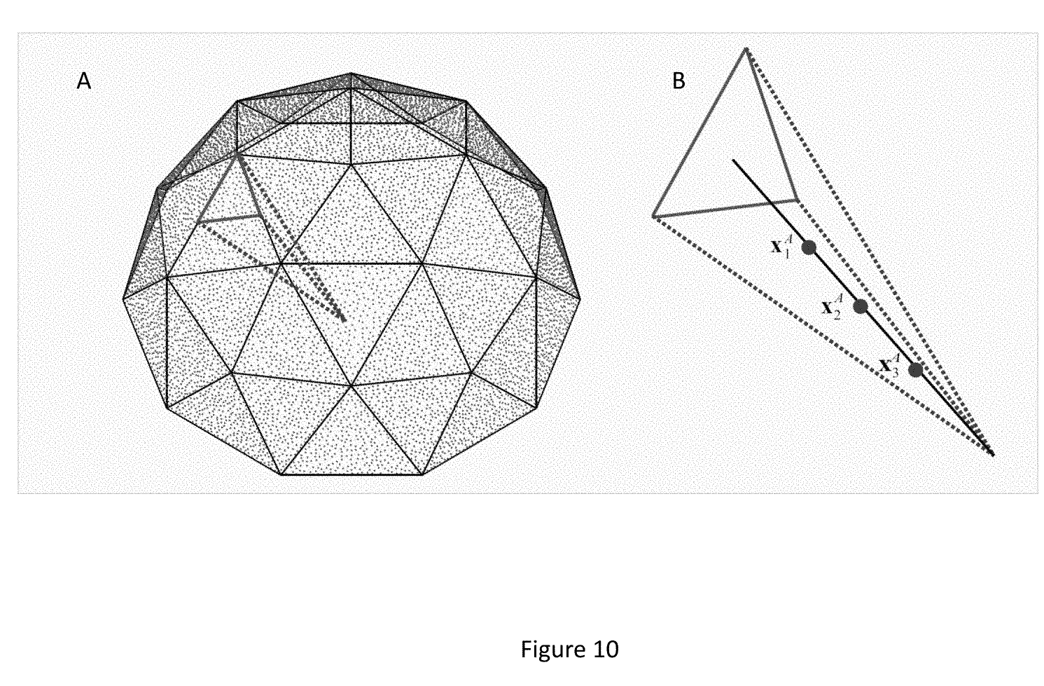

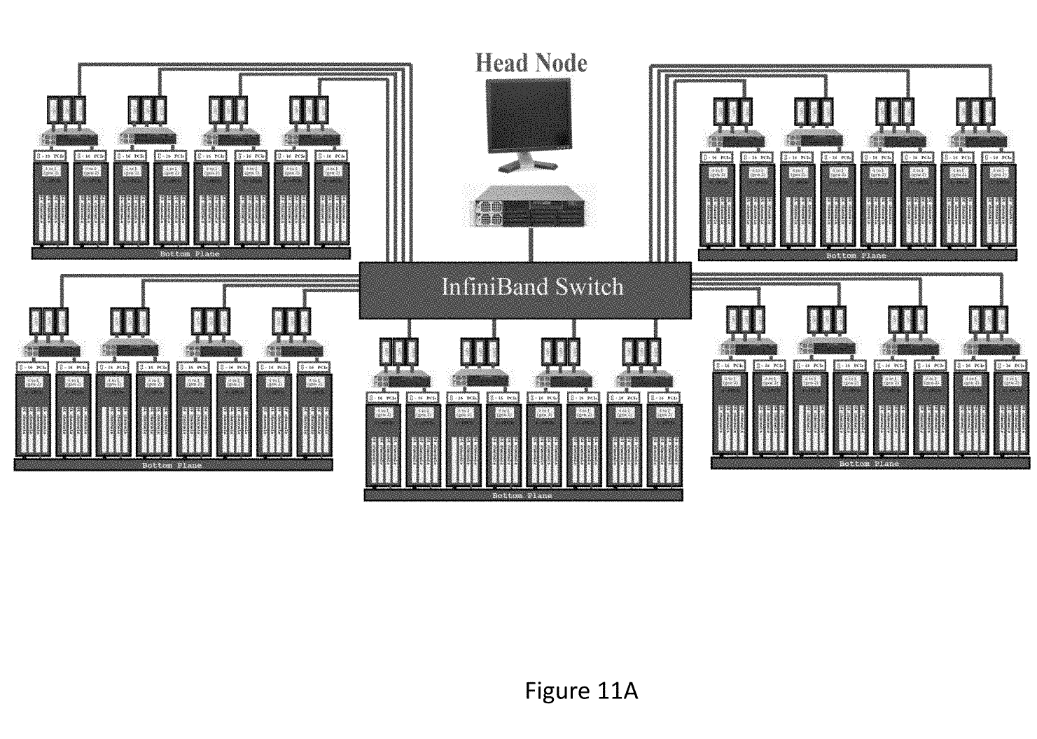

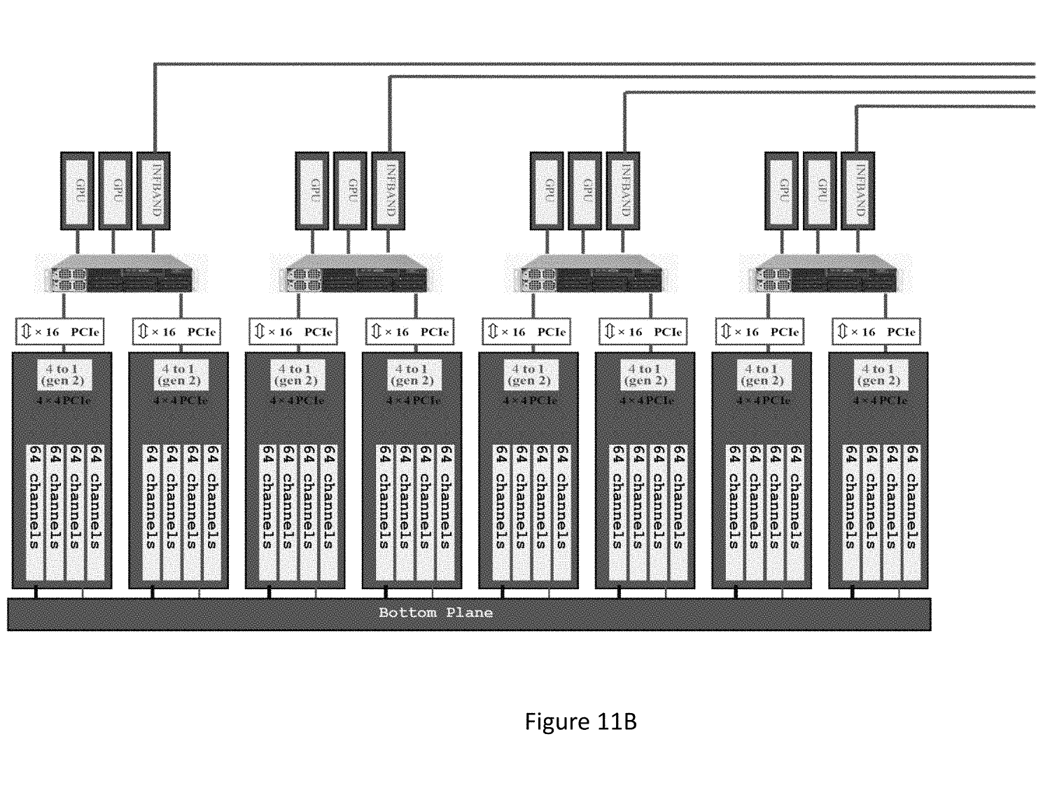

FIG. 11A is a diagram of an exemplary embodiment of a scalable computing network suitable to meet the processing needs for HABIS as described herein. For example, FIG. 11A shows the interconnections of a 21-node network that is comprised of 20 hybrid (acquisition and computation) nodes and a head (or control) node, which supervises the computing network. In this embodiment, the 40 data-acquisition boards are arranged in five groups of eight. Two data-acquisition boards are connected to each of the 20 computing nodes. The head node may also serve as a console for rendering images and for controlling the instrument. FIG. 11B shows a group of eight boards and their corresponding nodes in greater detail. The 20 computing nodes, each with two GPUs, form an efficient numerical engine for massively parallel computations. Single-precision GPUs are ideal computational engines for several reasons. Since the data acquired from the hemispheric transducer have 14 bits of precision, and since the computations applied to this data are not numerically unstable, single-precision arithmetic is sufficient. In fact, reduced memory requirements and faster transfer rates associated with single-precision data make this format desirable. Each reconstruction requires a very large number (about 50×10¹⁵ floating-point operations, i.e., 50 petaflops) of single-precision operations. Each node receives data from two of the 40 sets of data-acquisition electronics (shown as the green rectangles that contain four 64-channel blocks in FIG. 11B) via fast PCIe connections. Four GPUs are also connected to each node to form a powerful single-precision computational engine for parallel processing. InfiniBand connections to a central switch enable rapid inter-node communication necessary between stages of the computation. These switches allow 20 InfiniBand connections to be active simultaneously. In one embodiment, all the nodes of the network can also be connected to a local Ethernet switch (not shown) for command, control, and monitoring. A high-speed connection of the local Ethernet to the Internet further enables command, control, and monitoring from a remote location.

The reconstruction algorithm, which is described in detail later herein, can be parallelized at both a high and a low level. The high-level parallelization factors the computations into large-scale operations that are performed independently on separate nodes. The low-level parallelization reduces these operations to a succession of vector and matrix computations that can be implemented as GPU kernels. Also, only a modest amount of logic is needed to direct the flow of data and to sequence the computations. Careful sequencing of these kernels can use the asynchronous data transfer capability of currently available general-purpose GPUs to eliminate data transfer latencies, resulting in improved efficiency. Data-transfer latencies are further suppressed by generation 3 PCIe connections used in currently available general-purpose GPUs.

During an approximately two-second period of data acquisition that comprises a volume scan by HABIS, each of the 40 sets of data-acquisition electronics subassemblies receives 10,240 waveforms from 256 different transducer elements. For a sampling rate of 20 MHz over a time interval of 204.8 μs and for two bytes per sample, all of which are representative of those used in HABIS, a total of (2 bytes per sample)×(20 samples per μs)×(204.8 μs per waveform)×(10, 240 waveforms per element)×(256 elements)=21.47483×10¹⁰ bytes or about 21.5 gigabytes (GB) are acquired and stored by each set of electronics. As mentioned previously, if a separate computational facility were used for reconstruction, then the data from all 40 sets of the data-acquisition electronics (i.e., a total of 40×21.47483×1010 bytes=8.589934×10¹¹ bytes or about 0.859 TB) would need to be aggregated and transferred to that facility, a time-consuming process given the large amount of data to be transferred.

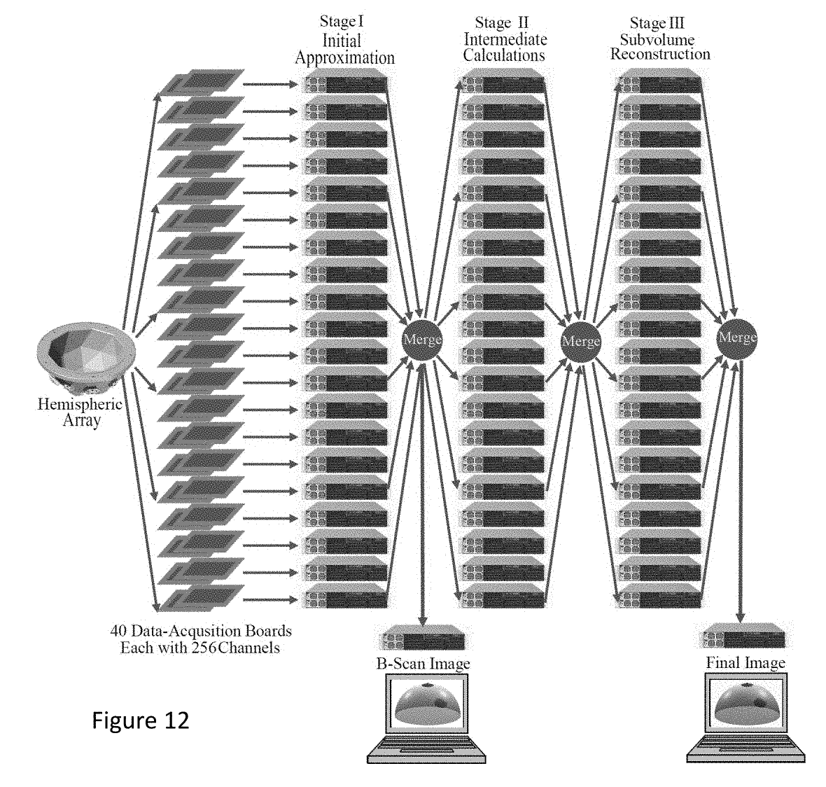

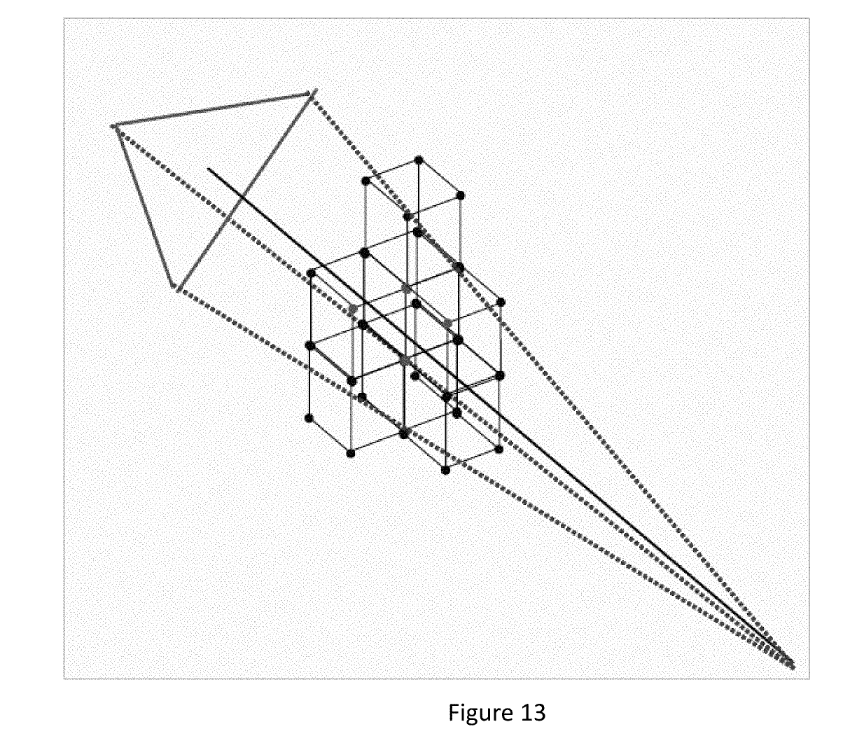

As contemplated herein, FIG. 12 illustrates an exemplary data processing flow (via the image reconstruction algorithm) that permits processing to be performed in stages that consist of parallel computations on each node. As shown, these parallel computations on one node may be completely independent of the computations on the other nodes. As shown in FIG. 12, Stage I operations include preprocessing steps (such as time-variable gain compensation, transmission decoding, and application time gates to isolate the response from different regions), b-scan imaging, and initial approximation of the scattering object. Stage II operations include split-step propagation of fields that emanate from each of the 10,240 transducer elements and pass through the approximate scattering object, reduction of these fields to simplified representations each limited to a single subvolume, and estimation of the portions of the scattering measurements attributable to the approximate scattering object. Stage III operations produce a refined approximation of the scattering object by using residual scattering measurements to reconstruct residual medium variations in each subvolume. After each stage of computations, the results of the computations at each node are redistributed to other nodes for the next stage of computations.

Computational efficiency and cost-effective implementation of the staged processing scheme described above were the main criteria used to design the high-performance computer network. Two key requirements follow from these criteria. One requirement is that the nodes must have enough computational capacity to complete each stage of computation in a timely manner. The other requirement is that the nodes must be able to exchange data rapidly enough so that data redistribution is not a bottleneck.

The computer nodes must also be endowed with enough memory to insure that the GPUs can be continuously supplied with input data, and also have enough storage for intermediate results so that saving and recalling data from disk files is unnecessary. In one embodiment, each HABIS compute node is equipped with four GPUs and a total of 128 GB of memory. These allocations allow each node to conduct four completely independent parallel computations during each processing stage. This combination of resources results in relatively low-cost compute nodes that have precisely the right kind of numerical capability for efficient implementation of the HABIS algorithm. In other embodiments, each HABIS compute node can include a different number of GPUs and/or memory, as would be understood by a person skilled in the art.

The transfer of data from the acquisition apparatus to the computer network and the node-to-node data transfers that occur after each stage of computation all involve large volumes of data. Each compute node in the architecture is responsible for collecting a receive signal of 4,096 two-byte temporal samples from a group of 512 receive channels for each of 10,240 transmissions. Based on the previously-noted volume of data for a 256-channel set, the total volume of all these samples is about 43 GB. Excluding overhead, this volume of data can be transferred over a fast 16-lane PCIe connection with an overall transfer rate of 8 GB/s in about 5.4 seconds and can be transferred over a pair of such PCIe connections in about 2.7 seconds.

Preprocessing occurs initially in each node without internode communication and consists of transmit-receive channel response equalization, compensation for time-varying gain, Fourier transformation, and signal decoding requires expansion of the two-byte samples to a larger size. In HABIS, although other expansions are possible, the expansion to a larger size is accomplished by conversion of the two-byte integer samples into four-byte floating-point values. This increases the volume of data associated with one node to 86 GB from 43 GB. Although Fourier transformation that is part of the preprocessing expands the four-byte floating-point values to complex values each occupying eight bytes, the volume of the complex data is orders of magnitude less if only a set of Fourier components in the useful bandwidth of the system are retained and the relation between positive and negative frequencies imposed by real temporal signals is used. In view of this, node-to-node data transfers that proceed subsequent processing involve smaller volumes of data.

The required node-to-node transfers can be performed quickly using InfiniBand connections instead of PCIe connections. The efficiency of InfiniBand connections is illustrated by considering the transfer of results from the parallel Stage II calculations, i.e., the scattering-measurement matrix \tilde{M} with entries \tilde{M}ₘₙ for the approximate scattering object. This matrix is comprised 10,240×10,240 complex numbers that are each eight bytes. Thus, the size of the entire matrix is about 0.84 GB, of which one-twentieth resides on each compute node. The aggregation of the entire matrix at each node for the Stage III computations requires a total transfer of

$${20}_{\overbrace{⏟}_{{Nodes that contain}_{a portion of {\tilde{M}}_{n m}}}} \times {0.84 GB / 20}_{\overbrace{⏟}_{{Size of each}_{portion of {\tilde{M}}_{n m}}}} \times {19}_{\overbrace{⏟}_{{Nodes to get}_{each portion}}} = 16 GB . \qquad Eq . ( 30 )$$

For a 4×QDR InfiniBand connection that, excluding overhead, has a data transfer rate of about 4 GB/s (i.e., 32 Gb/s), this transfer can be completed in four seconds using a single InfiniBand connection, and can be completed in about 0.2 seconds using 20 simultaneously-active InfiniBand connections.

### Communication and Control

The preprocessing and b-scan imaging that take place in Stage I are performed with no communication between the 20 nodes in the network. However, full inverse scattering reconstruction is completed by the Stage II and Stage III computations that require node-to-node transfers of data over the InfiniBand network. The 20 nodes also need to receive commands from an operator using a terminal to control the data collection, processing operations, b-scan image formation, and image reconstruction via inverse scattering. Communication software provides the necessary capabilities for data exchange, distributed control, and coordinated calculations among the nodes. Each of the 20 nodes includes a library that provides an interface to the other 19 nodes and a head node with a keyboard and monitor. From the head node, commands originated by an operator can be broadcast to the 20 computing nodes by using control program that runs on each node. The program can include logic that allows each node to determine its data-acquisition functions from the operator-originated requests. The program also supervises the preprocessing calculations. Additional programs that run on all the nodes form b-scan images and reconstruct images via inverse scattering. The terminal that accepts operator commands can be used as a user interface that runs on one of the 20 nodes or, alternatively, on the head node.

### Method and Software System for Hemispheric Array Breast Imaging

As described above, HABIS utilizes various embodiments of ultrasound imaging for improved detection, diagnosis, and monitoring for the recurrence of breast cancer. In certain embodiments, the system transmits ultrasound pulses into a breast and records received echoes. A parallel architecture allows all 10,240 ultrasound transducer elements to receive, without multiplexing, echoes from as many as 2,048 simultaneous transmissions. This architecture permits the collection of all required measurements in seconds. Once the data is recorded, it is sent to a high-performance computing cluster for imaging computations.

In various embodiments described above, 10,240 channels are divided among 40 front-end printed-circuit boards controlling 256 channels each. A 256-channel board is divided into four 64-channel groups. Each group includes 64 transducer interfaces, a single control FPGA, 8 GB of local DRAM for storage of measurements, and a 4-lane PCI Express (PCIe) interface. A PCIe switch on each 256-channel board aggregates the four channel-group interfaces into a single 16-lane PCIe interface. The 256-channel boards are connected via 16-lane, PCIe 2.0 extension cables to individual nodes of the high-performance cluster. Two boards are connected to each node.

A method for hemispheric array breast imaging according to one embodiment is embodied in three software systems: (1) FPGA firmware to control ultrasound transmissions and receptions as well as communication with the high-performance computing nodes, (2) low-level operating-system device driver that runs on the computing nodes to enable software interaction with the FPGA firmware, and (3) an application programming interface (API) library that abstracts the low-level representation of FPGA hardware by the device driver and provides input validation. These three systems are important to the operation of HABIS and are described in further detail below.

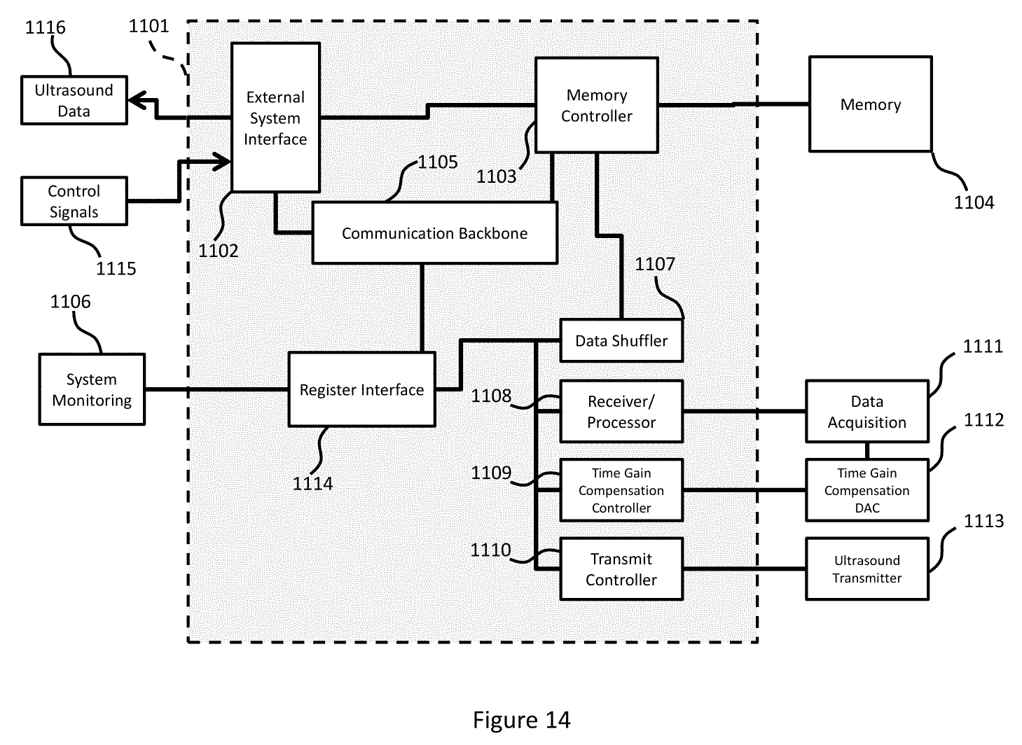

FIG. 14 depicts a system according to one embodiment for controlling multiple ultrasound transmitters, recording the data received from multiple ultrasounds receivers, and aggregating the received data and transmitting it to a high-performance computing node for analysis. In one embodiment, a processing unit 1101 is a field-programmable gate array (FPGA) which includes multiple interconnected logic cores. In certain embodiments, “logic core” or “core” refers to a collection of programmable logic elements whose collective behavior is defined by a hardware description language (HDL). A transmit controller core 1110 communicates with multiple ultrasound transmitters 1113 in order to generate pulsed ultrasound signals. The data acquisition modules 1111 in turn sample the returned pulses. The receiver/processor cores 1108 collect the samples and the data shuffler core 1107 aggregates the samples from the receiver/processor cores 1108. These aggregated samples are in turn stored in memory 1104. The memory controller 1103 provides an interface to the system memory 1104. The memory controller core 1104 receives read and write requests from the external system interface core 1102, the data shuffler core 1107, as well as other cores via the communication backbone 105. Each request comprises either a request to read data from a supplied memory address or a request to write data to a supplied memory address, as well as the data to write. The memory controller core 104 queues these requests and processes them in order. In the case of a read request, the memory controller core responds to the requesting core with the requested data.

The time-gain compensation (TGC) system includes the TGC Controller core 1109 and the TGC digital-to-analog converter (DAC) 1112. The TGC system adjusts for signal attenuation over time. Ultrasound signals decrease in amplitude as they travel through tissue, a phenomenon known as tissue attenuation. An echo received immediately from superficial tissue would have higher amplitude than an echo received later from identical, deeper tissue. An image constructed from such signals would present the deeper tissues as darker or less distinct, which is not ideal. The TGC system artificially increases the amplitude of signals received later so that the resultant image is more uniform.

Certain operating parameters of the Data Shuffler, Receiver/Processor, TGC Controller, and Transmit controller cores 1107, 1108, 1109, and 1110 are stored in and referenced from the register interface core 1114. The register interface core comprises a plurality of data values each given an address. For example, the number of ultrasound pulses transmitted per second might be specified in a register with address “05”. The transmit controller core 1110 would query address “05” in the register interface 1114 when determining how long to wait after sending an ultrasound pulse before sending another one. If the operator wanted to change the behavior of the ultrasound transmitter to increase or decrease the number of pulses per second, the operator could set register “05” to a different value using control signals 1115. The transmit controller core 1110 would then see this different value next time it queried register “05” and adjust its behavior accordingly.

The register interface also works in the opposite direction. For example, a data acquisition node 1111 might report the average received signal strength during an acquisition and store it in a register with address “06”. If the operator wanted to check the average received signal strength of that acquisition node without processing the full set of samples received, the operator could query the register interface 1114 with address “06” and quickly ascertain the average received signal strength. The register interface is also used to store data gathered from other parts of the system. For example, a system monitoring module 1106 might check the voltage levels of various power supplies elsewhere on a printed circuit board on which the processing unit 1101 is mounted. The operator could then check these values by querying the register interface with the appropriate address.

The external system interface 1102 provides a plurality of communication channels between the high performance computing node and the processing unit 1101. In addition to providing the high performance computing node with the ultrasound data 1116, the external system interface 1102 is also the method by which the operator sends control signals 1115 to the various logic cores in the processing unit 1101.

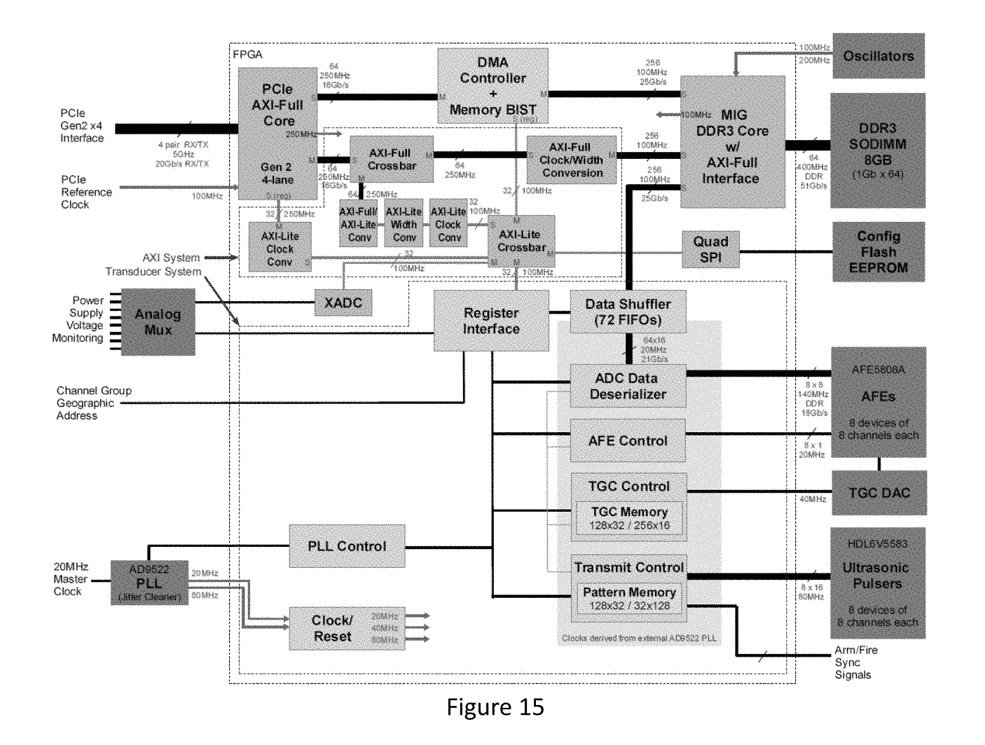

An exemplary embodiment of the firmware is shown in FIG. 15. Blocks shaded green represent custom logic developed for each 256-Channel FPGA. Blocks shaded purple represent modules based on Xilinx IP cores, and blocks shaded blue represent external devices that interface with the FPGA. The blocks grouped in brown represent custom control modules directly related to the operation of the ultrasound transducers. The remaining blocks within the FPGA are general-purpose modules related to storage and transmission of data between the computing nodes and the 8-GB DRAM module as well as timing control. The FPGA modules are controlled and monitored by adjusting or reading values from a bank of registers. Software access to the registers is provided by the device driver and API.

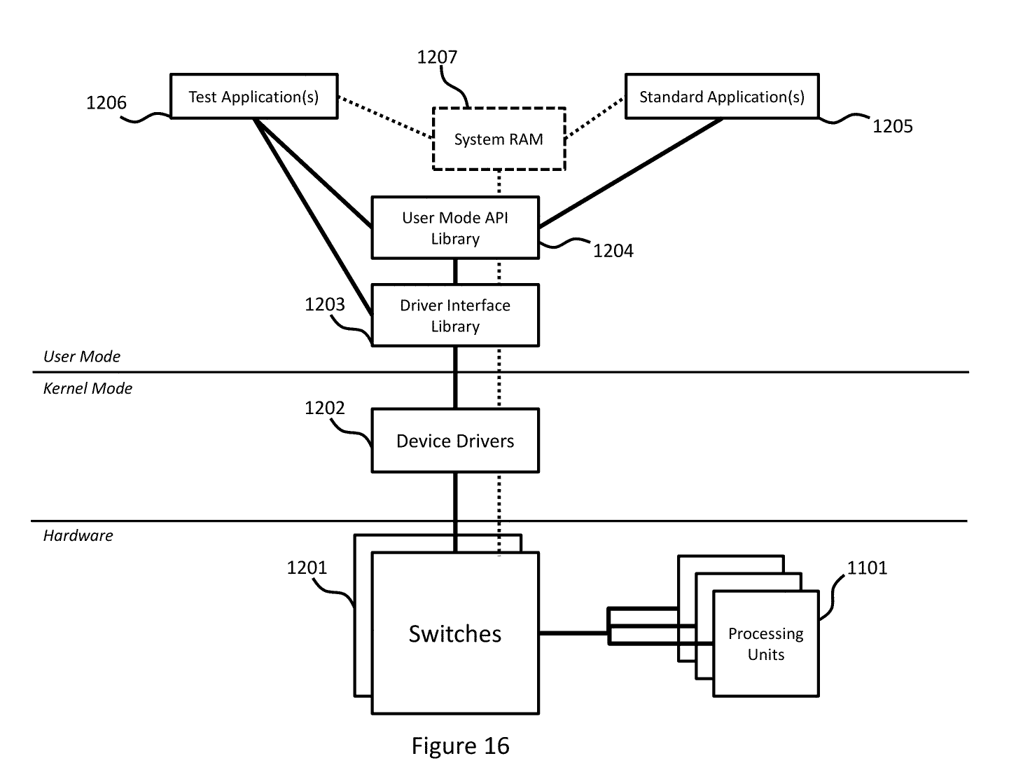

FIG. 16 depicts a schematic according to one embodiment of a library and driver software stack. The different modules are shown divided generally into three groups: User Mode, Kernel Mode, and Hardware. In certain embodiments, User Mode Software runs in User Space on a computer system, and in certain embodiments the software that must access system memory via the operating system. In certain embodiments, Kernel Mode Software runs in Kernel Space on a computer system, and in certain embodiments the software may directly access system memory without relying on the operating system. The individual processing units 1101 are connected via a bus to a series of switches 1201, which combine the data from the various processing units and communicate them to the high-performance computing node. In certain embodiments, the bus referred to herein is a logical bus which encompasses any physical interconnect for which devices and locations are accessed by an address. In one embodiment, the external system interface 1102 of each processing unit 1101 is a four-lane PCI Express bus, and each of the hardware switches 1201 combines four four-lane PCI Express buses into a single 16-lane PCI Express bus for communication with the device drivers 1202.

The device drivers 1202 provide a very low-level representation of the processing unit hardware to the high-performance computing node. The device driver runs within kernel space and provides identification within the computer system's device hierarchy for each of the attached processing units 1101. Additionally, the device driver allows reading and writing of individual registers 1114 on the processing unit, and reading and writing to the dedicated memory 1104 of each processing unit. In one embodiment, the device driver reads and writes from the individual registers 1114 using UNIX Input/Output Control (IOCTL) methods. In one embodiment, the device driver 1202 facilitates a connection between individual processing unit memory locations 1104 and the system memory 1207 of the high-performance computing node using Direct Memory Access (DMA).

The driver interface library 1203 provides the lowest-level user mode interface in the software stack on the high-performance computing node. In certain embodiments, “software stack” refers to the interconnected layers of software running on the high-performance computing node. In the preferred embodiment, the driver interface library provides a wrapper around the IOCTL functions and does some input validity checking, error detection and handling, and logging for debugging and development. Though test applications 1206 might directly call functions exposed by the driver interface library 1203, standard applications 1205 are not expected to communicate with the device drivers at such a low level of abstraction.

The user-mode Application Programming Interface (API) library 1204 provides a collection of function calls to applications 1205 and 1206. Applications may use the exposed function calls to configure and control individual processing units 1101, as well as to retrieve the captured ultrasound measurement data. In one embodiment, the API software 1204 is written in C++ and is object-oriented, but it is possible to implement an API in any computer language. The API library 1204 is written at a higher abstraction level than the driver interface library 1203.

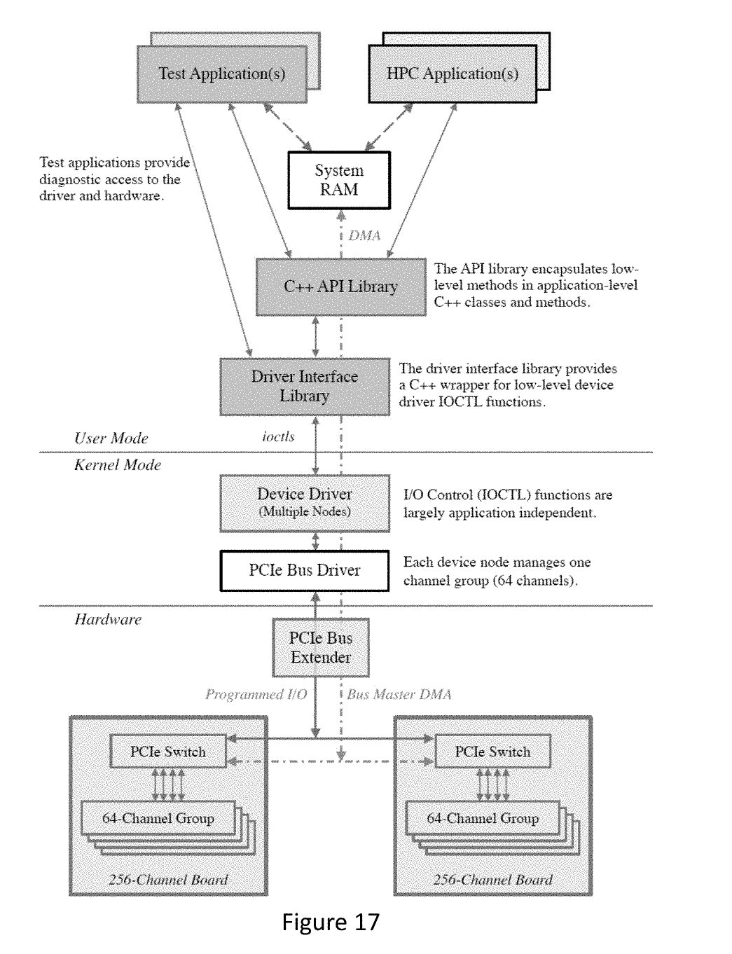

An exemplary implementation of a HABIS API library and driver software stack is shown in FIG. 17. The HABIS API library provides a collection of C++ classes to be used by application programs running on each computing node to configure and control the HABIS hardware and to transfer the captured measurement data. The API is built on top of a driver interface library, but the higher abstraction level of the API library allows it to implement additional validation and error handling that would not be feasible in the lower-level interface.

The driver interface library is the lowest level user-mode software in the HPC software stack. This library consists of a lightweight C++ object-oriented wrapper around the IOCTL functions implemented by the kernel device driver. This library does not significantly alter the abstraction level presented by the device driver. The methods exposed by driver interface classes implement argument validity checking, error detection and handling, and support for debugging and development (e.g., logging). Application-level programs are not expected to directly use the driver interface library.

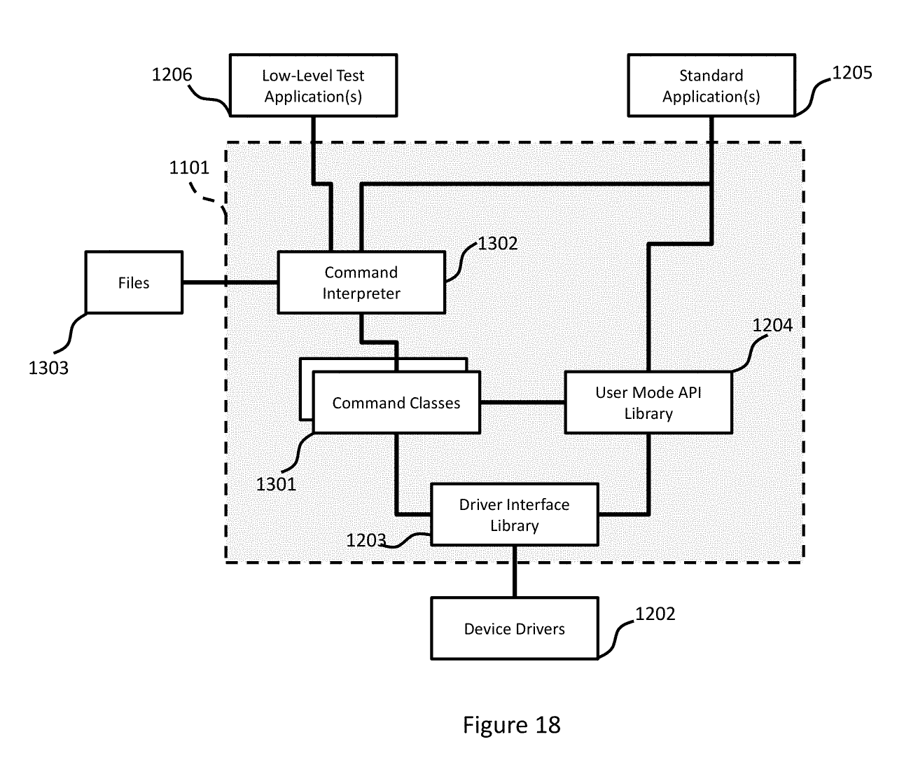

FIG. 18 depicts the internal structure of the HABIS API library and software stack 1202, 1203, and 1204 according to one embodiment. This embodiment adds command-handler classes that handle pre-defined messages from a command interpreter 1302. The command interpreter accepts commands from applications 1205, 1206 or from a list contained in a file 1303. In one embodiment, a test application may issue a command or set of commands in order to test one part or a subset of the system. In another embodiment, standard applications 1205 may load a pre-defined command set from a file 1303 in order to perform a procedure repeatably. In one embodiment, a plurality of processing units 1101 are disposed on a single printed circuit board (PCB), and each processing unit controls multiple ultrasound transducers. In one embodiment, each processing unit 1101 controls 64 ultrasound transducers, and four processing units share a single PCB, for a total of 256 ultrasound channels per PCB.

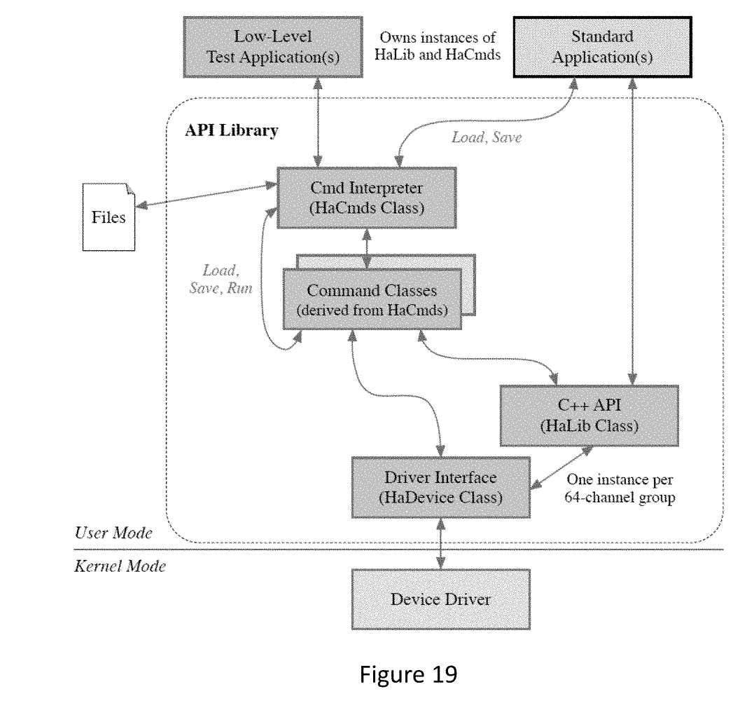

An exemplary implementation of the API library software stack is shown in FIG. 19 according to one embodiment. The main API library class, HaLib, is used by standard applications as well as by low-level test and diagnostic applications. One instance of the HaLib class is created for each attached 64-channel group device. The API library also provides a collection of command-interpreter classes, HaCmds, to provide intermediate-level control. The HaCmds class hierarchy presents a view into low-level device operation that can be used by diagnostic and test applications to confirm proper operation of the device. In addition, HaCmds can be used by standard applications to save and load device control sequences that together constitute higher-level conceptual operations.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

### Illustration of HABIS Imaging Capabilities

Reconstructions based on inverse scattering and corresponding b-scan images have been obtained using calculated scattering by a 10-point resolution target, and by a realistic breast model derived from 200-μm resolution MRI data to illustrate the resolution and fidelity of the volumetric images produced by HABIS. Each of the inverse-scattering reconstructions used scattering at the surface of the entire hemispheric transducer array, and also used an estimation of scattering object detail that would come from measurements in the opposite hemisphere where scattering cannot be directly measured. This approach obtains spatial frequencies spanning an entire lowpass volume in the Fourier transform of the image space (a transform space known as wave space) to realize the maximum theoretical amount of spatial detail contained in that volume. Each of the b-scans used transmission from a 64-element subtriangle and three 64-element subtriangles surrounding that central subtriangle, and used reception from the central 64 element subtriangle. Use of these transmit and receive apertures takes advantage of the system architecture to allow formation of a volumetric b-scan in about one minute after a two-second interval of data collection. This b-scan has sufficient resolution to assess the quality of the acquired data for a more time-consuming volumetric reconstruction based on inverse scattering.

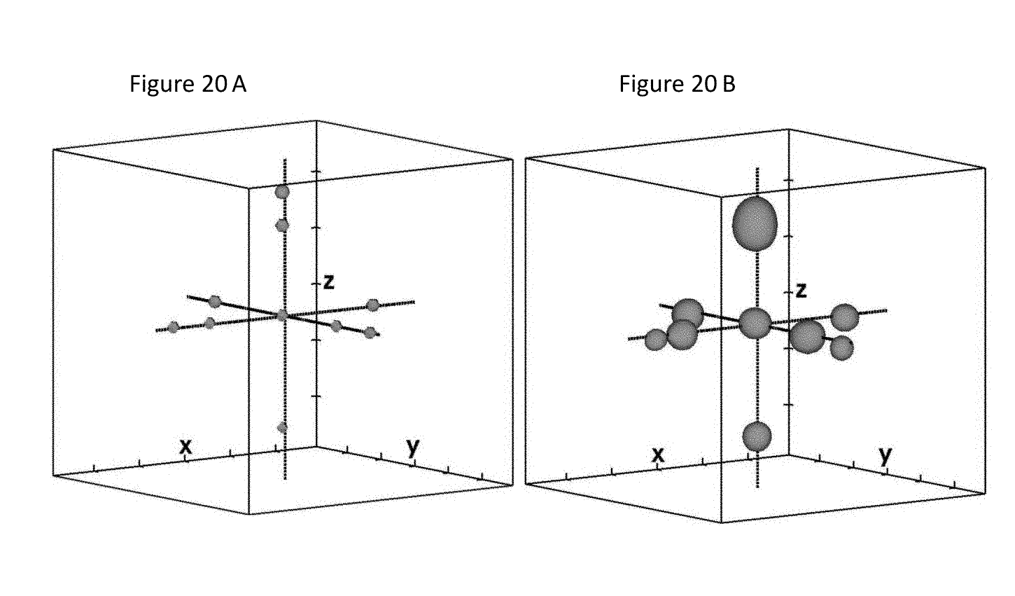

Representative images of the resolution target are shown in FIGS. 20A and 20B. At the 5-MHz frequency used to obtain FIG. 20A, the Rayleigh-defined resolution inferred from inverse-scattering reconstructed data is 100 μm. For the location of the resolution target in the b-scan shown in FIG. 20B, the transmit f-number was about 1.5 and the receive f-number was about 2.9. The pulse spectrum used for the b-scan had a raised-cosine shape centered around 5 MHz with a −6-dB bandwidth of 2 MHz. These transmit-receive apertures and pulse bandwidth yield an isotropic Rayleigh-defined resolution of about 700 μm. A b-scan using transmission and reception throughout the hemisphere would have a lateral resolution comparable to the resolution in the reconstruction as a result of the transmit and receive effective f-numbers each being one half.

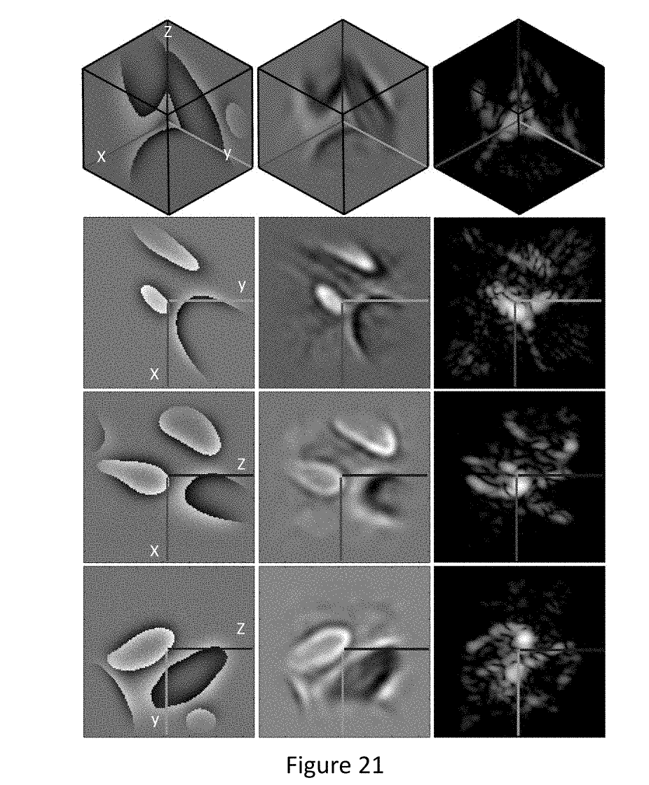

Representative sections of a 6.4-mm3 subvolume in the breast model are shown in FIG. 21. The reconstruction was performed at a frequency of 5 MHz as in the case of the resolution target. The detail evident in the reconstructed-image panel of FIG. 21 comes from the use of scattering throughout the entire surface of the hemispheric array. This scattering was first used to estimate scattering measurements that would be made by a virtual array in the opposite hemisphere and then the combined set of data was used in the reconstruction. The b-scan used aberration correction. For the location of the subvolume of the model, the transmit f-number in the b-scan was about 3.0 and the receive f-number in the b-scan was about 6.0. These transmit-receive apertures yield a Rayleigh-defined lateral resolution of about 1.5 mm at the 5-MHz center frequency. The pulse spectrum was the same as used for the resolution target so the axial resolution is about 700 μm as in the case of the resolution target. The inverse-scattering reconstruction clearly shows the subvolume morphology with high fidelity while the b-scan mainly highlights discontinuities that are approximately perpendicular to the b-scan lines.

What is claimed is:

1. A device for volumetric ultrasound imaging comprising:
an array of transducer elements substantially configured in the shape of a hemisphere to form a cup-shaped volumetric imaging region within the cavity of the hemisphere; and
a control module comprising a firmware module, a low-level operating-system device driver and an application programming interface library for processes ultrasound signals transmitted and received from the array of transducer elements.

2. The device of claim 1, wherein the firmware module is an FPGA firmware module configured to control ultrasound transmissions and receptions, and communicate with a plurality of computing nodes.

3. The device of claim 1, wherein the low-level operating-system device driver is configured to run on the computing nodes to enable software interaction with the firmware module.

4. The device of claim 1, wherein the application programming interface library abstracts the low-level representation of FPGA hardware by the device driver and provides input validation.

5. The device of claim 1, wherein the array of transducers comprises 40 triangular planar facets.

6. The device of claim 5, wherein 10 of the facets are equilateral triangles and 30 of the facets are isosceles triangles.

7. The device of claim 5, wherein each facet comprises 256 piezoelectric elements.

8. The device of claim 1, wherein at least one of the transducers further comprises a diverging lens.

9. The device of claim 1, wherein at least one of the transducers further comprises two matching layers.

10. The device of claim 1, wherein the hemisphere array of transducers is positioned within the surface of a patient table, such that the opening of the cup-shaped volumetric imaging region is substantially flush with the patient table surface.

11. A system for volumetric ultrasound imaging, comprising:
an array of planar faceted ultrasound transducers substantially configured in the shape of a hemisphere to form a cup-shaped volumetric imaging region within the cavity of the hemisphere;
a plurality of data-acquisition assemblies connected to the transducers;
a network of processors connected to the data-acquisition assemblies; and
a control module comprising a firmware module, a low-level operating-system device driver and an application programming interface library for processing ultrasound signals transmitted and received from the array of transducers;
wherein the ultrasound transducers are configured to generate and receive ultrasound signals within the imaging region, the data-acquisition assemblies are configured to collect ultrasound signals received from the transducers and transmit image data to the network of processors, and the network of processors is configured to construct a volumetric image of an object within the imaging region based on the image data received from the data-acquisition assemblies.

12. The system of claim 11, wherein the firmware module is an FPGA firmware module configured to control ultrasound transmissions and receptions, and communicate with a plurality of computing nodes.

13. The system of claim 11, wherein the low-level operating-system device driver is configured to run on the computing nodes to enable software interaction with the firmware module.

14. The system of claim 11, wherein the application programming interface library abstracts the low-level representation of FPGA hardware by the device driver and provides input validation.

15. The system of claim 11, wherein the number of data-acquisition assemblies is equal to the number of transducers, and that each data-acquisition assembly is dedicated to an individual transducer.

16. The system of claim 15, wherein the array of transducers comprises 40 triangular planar faceted transducer subarrays.

17. The system of claim 16, wherein 10 of the facets are equilateral triangles and 30 of the facets are isosceles triangles.

18. The system of claim 11, wherein the network of processors comprises at least 20 nodes.

19. The system of claim 18, wherein each node comprises at least one graphical processing unit (GPU).

20. The system of claim 19, wherein each node is configured to process data received from at least two data-acquisition assemblies in parallel.
